(19) United States
(12) Patent Application Publication
(10) Pub. No.: US 2008/0234300 A1
(43) Pub. Date: Sep. 25, 2008

(54) Pyrimido[5,4-c] Quinoline-2, 4-Diamine Derivatives and Methods of Use Thereof

(75) Inventors: Allan WISSNER, Ardsley, NY (US); Aranapakam M. VENKATESAN, Regopark, NY (US)

Correspondence Address: WYETH;PATENT LAW GROUP, 5 GIRALDA FARMS, MADISON, NJ, 07940, US

(73) Assignee: WYETH, Madison, NJ (US)

(21) Appl. No.: 12/042,092

(22) Filed: Mar. 4, 2008

Related U.S. Application Data
(60) Provisional application No. 60/904,945, filed on Mar. 5, 2007.

(51) Int. Cl. A61K 31/519; C07D 471/04; A61P 35/00

(52) U.S. Cl. 514/267; 544/250

(57) ABSTRACT

The present invention relates to pyrimido[5,4-c]quinoline-2,4-diamine derivatives, compositions comprising an effective amount of a Pyrimido[5,4-c]quinoline-2,4-diamine Derivative, methods for treating or preventing a proliferative disorder, comprising administering to a subject in need thereof an effective amount of an Pyrimido[5,4-c]quinoline-2,4-diamine Derivative, methods for modulating PDK-1 activity, comprising administering to a subject in need thereof an effective amount of a Pyrimido[5,4-c]quinoline-2,4-diamine Derivative. The invention also relates to a process for preparing a Pyrimido[5,4-c]quinoline-2,4-diamine Derivative.

### 1. REFERENCE TO RELATED APPLICATIONS

This application claims priority to U.S. Provisional Application No. 60/904,945, filed Mar. 5, 2007, the disclosure of which is incorporated by reference herein in its entirety.

### 2. FIELD OF THE INVENTION

The present invention relates to Pyrimido[5,4-c]quinoline-2,4-diamine Derivatives, compositions comprising an effective amount of a Pyrimido[5,4-c]quinoline-2,4-diamine Derivative and methods of treating or preventing a proliferative disorder, comprising administering a subject in need thereof an effective amount of a Pyrimido[5,4-c]quinoline-2,4-diamine Derivative.

### 3. BACKGROUND OF THE INVENTION

Cancer is second only to cardiovascular disease as the leading cause of death in the United States. The American Cancer Society estimated that 1.4 million new cancer cases would be diagnosed and 565,000 people would die of cancer in 2006 (American Cancer Society, Cancer Facts and Figures 2006, Atlanta, Ga.). The National Cancer Institute estimated that in January 2002, approximately 10.1 million living Americans had a history of cancer. The National Institutes of Health estimate direct medical costs of cancer as over $100 billion per year with an additional $100 billion in indirect costs due to lost productivity—the largest such costs of any major disease.

Cancer is a process by which the controlling mechanisms that regulate cell growth and differentiation are impaired, resulting in a failure to control cell turnover and growth. This lack of control causes a tumor to grow progressively, enlarging and occupying space in vital areas of the body. If the tumor invades surrounding tissue and is transported to distant sites, death of the individual can result.

The selective killing of cancer cells, while minimizing deleterious effects on normal cells, is a desired goal in cancer therapy. Modalities commonly used in the treatment of cancer include chemotherapy, radiation therapy, surgery and biological therapy (a broad category that includes gene-, protein- or cell-based treatments and immunotherapy). Despite the availability of a variety of anticancer agents, traditional chemotherapy has drawbacks. Many anticancer agents are toxic, and chemotherapy can cause significant, and often dangerous, side effects, including severe nausea, bone marrow depression, liver, heart and kidney damage, and immunosuppression. Additionally, many tumor cells eventually develop multi-drug resistance after being exposed to one or more anticancer agents. As such, single-agent chemotherapy is effective for only a very limited number of cancers. Many chemotherapeutic drugs are anti-proliferative agents, acting at different stages of the cell cycle. Because it is difficult to predict the pattern of sensitivity of a neoplastic cell population to anticancer drugs, or the current stage of the cell cycle that a cell happens to be in, it is common to use multi-drug regimens in the treatment of cancer.

Despite the significant research efforts and resources which have been directed towards the development of novel anticancer agents and improved methods for treating cancer, there remains a need in the art for novel compounds, compositions, and methods that are useful for treating cancer with improved therapeutic indices.

The interaction of insulin and growth factors with their receptors on the outside surface of a cell, leads to the activation of phosphatidylinositol 3-kinase (PI 3-kinase) and generation of the phosphatidylinositol 3,4,5-trisphosphate (PtdIns(3,4,5)P₃) second messenger at the inner surface of the cell membrane. One of the most studied signaling events controlled by PtdIns(3,4,5)P₃, comprises the activation of a group of cAMP-dependent protein kinase/protein kinase G/protein kinase C (AGC) family protein kinases, including isoforms of protein kinase B (PKB)/Akt, p70 ribosomal S6 kinase (S6K), serum- and glucocorticoid-induced protein kinase (SGK) and protein kinase C (PKC), which are involved in regulating physiological processes relevant to metabolism, growth, proliferation and survival. Studies have shown that 3-phosphoinositide-dependent protein kinase-1 (PDK-1) phosphorylates and activates the AGC kinase members regulated by PI 3-kinase (see, e.g., Mora et al., Semin Cell Dev Biol (2004) 15:161-170).

There remains a need in the art for novel compounds, compositions and methods that are useful for inhibition or modulation of PDK-1 activity.

Citation of any reference in Section 2 is not an admission that the reference is prior art.

### 4. SUMMARY OF THE INVENTION

In one aspect, the invention provides a compound of Formula (I)

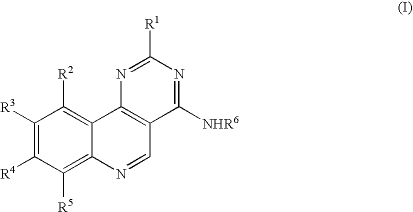

and pharmaceutically acceptable salts thereof,
wherein

R¹ is —H, —N(R¹²)₂, —R¹³, —NR¹²—(C(R¹²)₂)ₙ—OR¹², —NR¹²—(C(R¹²)₂)ₙ—H, —NR¹²—(C(R¹²)₂)ₙ—R¹³, —NR¹²—(C(R¹²)₂)ₙ—N(R¹²)₂, —NR¹²—(C(R¹²)₂)ₙ—NR¹²—(C(R¹²)₂)ₙ—N(R¹²)₂, NR¹²—(C(R¹²)₂)ₙ—NR¹²—(C(R¹²)₂)ₙ—OR¹²;

n=1-4 at each occurrence, with the proviso that when the carbon chain of length n is between two heteroatoms, n=2-4;

R² and R⁵ are each independently —H, —OH, -halo, —CN, —N₃, —NH₂, —NH—C₁-C₆ alkyl, —N(C₁-C₆ alkyl)₂, —C₁-C₆ alkyl, —O—C₁-C₆ alkyl, or —S—C₁-C₆ alkyl, wherein each C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R³ and R⁴ are each independently —H, —OH, -halo, —NH₂, —CN, —NO₂, —COOH, —C₁-C₆ alkyl, —C₂-C₆ alkenyl, —C₂-C₆ alkynyl, —O—C₁-C₆ alkyl, —O—C₂-C₆ alkenyl, —O—C₂-C₆ alkynyl, —C(O)O—C₁-C₆ alkyl, —C(O)NH—C₁-C₆ alkyl, —C(O)N(C₁-C₆ alkyl)₂, —C₁-C₆-alkylene-O—C₁-C₆ alkyl, —O—C₁-C₆-alkylene-O—C₁-C₆ alkyl, —C₁-C₆-alkylene-C(O)O—C₁-C₆ alkyl, —C₁-C₆-alkylene-C(O)NH—C₁-C₆ alkyl, —OC(O)—C₁-C₆ alkyl, —OC(O)—C₂-C₆ alkenyl, —OC(O)—C₂-C₆ alkynyl, —C₁-C₆ alkylene-OC(O)—C₁-C₆ alkyl, —C₁-C₆ alkylene-OC(O)—C₂-C₆ alkenyl, —C₁-C₆ alkylene-OC(O)—C₂-C₆ alkynyl, —S—C₁-C₆ alkyl, —S(O)—C₁-C₆ alkyl, —S(O)₂—C₁-C₆ alkyl, —S(O)₂NH—C₁-C₆ alkyl, —S(O)₂NH—C₂-C₆ alkenyl, —S(O)₂NH—C₂-C₆ alkynyl, —C(O)-benzyl, —C(O)O—C₁-C₆ alkyl, —C(O)—C₁-C₆ alkyl, -phenyl, —O-phenyl, —S-phenyl, -benzyl, —O-benzyl, —CHO, —NHC(O)H, —NHOH, —NH—O—C₁-C₆ alkyl; —NH—C₁-C₆ alkyl, —N(C₁-C₆ alkyl)₂, —NHC(O)—C₁-C₆ alkyl, —N(C₁-C₆ alkyl)(C₂-C₆ alkenyl), —N(C₂-C₆ alkenyl)₂, —NH-phenyl, —NH-benzyl,

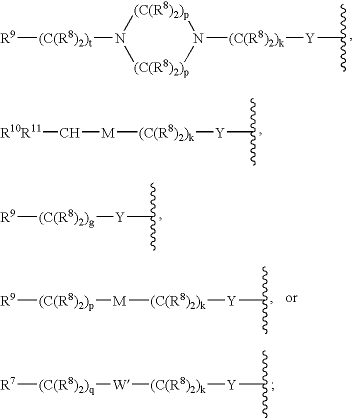

wherein

a=0-1;

g=1-6;

k=0-4;

p=2-4 at each occurrence;

q 0-4;

t=2-4;

M is a bond, —NR⁸—, —O—, —N((C(R⁸)₂)ₚN(R₈)₂)—, —N((C(R⁸)₂)ₚOR⁸)—, —CC—, —CH═CH—, or phenylene;
W′ is a bond, —N(R⁸)—, —O—, —CC—, —CH═CH—, or phenylene;
Y is —(CH₂)ₐ—, —O—, —S—, —C(O)N(R⁸)—, —C(S)N(R⁸), or —NR⁸—;
when M is phenylene then p=0-4 and r=0-4;
when M is —O— and R⁹ is —OR⁸ then p=1-4;
when M is —CC— or —CH═CH— and Y is —S—, —O— or —NR⁸— then k=1-4;
when M is —CC— or —CH═CH— then p=1-4;
when W′ is —CC— or —CH═CH— and R⁷ is bonded through a heteroatom then q=1-4;
when W′ is a bond and q=0 and R⁷ is bonded through a N atom and Y is —S—, —O— or —NR⁸— then k=2-4;
when W′ is a bond and k=0 and R⁷ is bonded through a N atom and Y is —S—, —O— or —NR⁸— then q=2-4;
when W′ is not a bond, —CC—, —CH═CH—, or phenylene and R⁷ is bonded through a N atom then q=2-4;
when Y is —NR⁸— and R⁹ is —N(R⁸)₂, —N(R⁸)₃⁺ or —NR⁸(OR⁸) then g=2-6;
when Y is —N(R⁸)— and M is —N(R⁸)—, —O—, —N((C(R⁸)₂)ₚN(R⁸)₂— or —N((C(R⁸)₂)ₚ—OR⁸)— then k=2-4;
when Y is —N(R⁸)— and W′ is —N(R⁸)— or —O— then k=2-4;
when Y is —O— and either M or W′ is —O— then k=1-4;
   wherein each —C₁-C₆ alkyl, —C₁-C₆ alkylene, —C₂-C₆ alkenyl, —C₂-C₆ alkynyl, -phenyl, -benzyl, or phenylene is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R⁶ is —H, —OH, or —C₁-C₆ alkyl, wherein each —C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R⁷ is -phenyl, a 5- or 6-membered aromatic monocyclic heterocycle which is unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl; or

R⁷ is a 3- to 7-membered non-aromatic monocyclic heterocycle which is unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl, benzoyl, -oxo or -thio,

wherein each R⁷ is independently unsubstituted or mono or di-substituted on a C atom with —R⁸, —N(R⁸)₂, —OR⁸, —(C(R⁸)₂)ᵣOR⁸, or —(C(R⁸)₂)ᵣN(R⁸)₂, or a 3- to 7-membered monocyclic heterocycle,

wherein r=1-6; or

each R⁷ is independently unsubstituted or mono or di-substituted on a saturated C atom with —O(C(R⁸)₂)ᵣO—,

wherein r=1-6; or

each R⁷ is independently unsubstituted or mono-substituted on a N atom with —R⁸;

R⁸ is each independently —H, —C₁-C₆ alkyl, —C₂-C₆ alkenyl, —C₂-C₆ alkynyl, —C₃-C₈ monocyclic cycloalkyl, —C(O)—C₁-C₆ alkyl, —C(O)O—C₁-C₆ alkyl, or -phenyl, wherein each —C₁-C₆ alkyl —C₂-C₆ alkenyl, —C₂-C₆ alkynyl, —C₃-C₈ monocyclic cycloalkyl or -phenyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R⁹ is -halo, —N(R⁸)₂, —OR⁸, —N(R⁸)₃⁺, or —NR⁸(OR⁸);

R¹⁰ and R¹¹ are each independently —(C(R⁸)₂)ₛN(R⁸)₂ or —(C(R⁸)₂)ₛOR⁸,

wherein s=1-4;

R¹² is each independently —H, —C₁-C₆ alkyl, —C₂-C₆ alkenyl, —C₂-C₆ alkynyl, —C₃-C₈ monocyclic cycloalkyl, —C₃-C₈ monocyclic cycloalkenyl, -phenyl, -benzyl, a 5- or 6-membered aromatic monocyclic heterocycle, or a 3- to 7-membered non-aromatic monocyclic heterocycle which is unsubstituted or substituted with one or more of —OH, -halo, —NH₂, —CN, —C₁-C₆ alkyl, -oxo or -thio, wherein each —C₁-C₆ alkyl, —C₂-C₆ alkenyl, —C₂-C₆ alkynyl, —C₃-C₈ monocyclic cycloalkyl, -phenyl, or benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R¹³ is a 5- or 6-membered aromatic monocyclic heterocycle which is unsubstituted or substituted with one or more of —OH, -halo, —NH₂, —CN or —C₁-C₆ alkyl, a 3- to 7-membered non-aromatic monocyclic heterocycle which is unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl, benzoyl, -oxo or -thio, or a 8- to 12-membered bicyclic heterocycle which is unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl, benzoyl, -oxo or -thio,

wherein each R¹³ is independently unsubstituted or mono- or di-substituted with 4-(2,3-dihydro-indol-1-yl), 4-(1,3-dihydro-isoindol-2-yl), —NH-benzoyl, a 5- or 6-membered aromatic monocyclic heterocycle, a 3- to 7-membered non-aromatic monocyclic heterocycle, 8- to 12-membered bicyclic heterocycle, —O-5- or 6-membered aromatic monocyclic heterocycle, —O-3- to 7-membered non-aromatic monocyclic heterocycle, —O-8- to 12-membered bicyclic heterocycle, or R¹⁴,

wherein each -benzoyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R¹⁴ is —H, -halo, —OH, —CN, —NH₂, —NO₂, —N₃, —COOH, —C₁-C₆ alkyl, —C₂-C₆ alkenyl, —C₂-C₆ alkynyl, —N₃, —NHC(O)—C₁-C₆ alkyl, —C₁-C₆ alkyl —O—C₁-C₆ alkyl, —C₁-C₆ alkylene-C(O)O—C₁-C₆ alkyl, —O—C₁-C₆ alkyl, —S—C₁-C₆ alkyl, —C(O)O—C₁-C₆ alkyl, —C(O)—C₁-C₆ alkyl, -phenyl, —O-phenyl, —S-phenyl, -benzyl, -benzoyl, —C₁-C₆ alkylene-C(O)O-phenyl, —NH—C₁-C₆ alkyl, —N(C₁-C₆ alkyl)₂, —NH-phenyl, —NH-benzyl, —NH—C(O)—C₁-C₆ alkyl, —NH—C(O)—C₂-C₆ alkenyl, —NH—C(O)—C₂-C₆ alkynyl, —C₁-C₆ alkylene-COOH, —C₁-C₆ alkylene-CHO, —C₁-C₆ alkylene-NH—C₁-C₆ alkyl, —C₁-C₆ alkylene-N(C₁-C₆ alkyl)₂, —C(O)NH—C₁-C₆ alkyl, —C(O)N(C₁-C₆ alkyl)₂, —O—C₁-C₆ alkylene-NH—C₁-C₆ alkyl, —O—C₁-C₆ alkylene-N(C₁-C₆ alkyl)₂, —OS(O)₂—C₁-C₆ alkyl, or —SH, wherein each —C₁-C₆ alkyl, —C₁-C₆ alkylene, —C₂-C₆ alkenyl, —C₂-C₆ alkynyl, -phenyl, -benzyl, or benzoyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl.

The invention provides compositions comprising an effective amount of a compound of Formula (I) or a pharmaceutically acceptable salt thereof and a physiologically acceptable carrier or vehicle.

A compound of Formula (I) or a pharmaceutically acceptable salt thereof (a “Pyrimido[5,4-c]quinoline-2,4-diamine Derivative”) is useful for treating or preventing a proliferative disorder.

A composition comprising an effective amount of a Pyrimido[5,4-c]quinoline-2,4-diamine Derivative is useful for treating or preventing a proliferative disorder.

The invention further provides methods for treating or preventing a proliferative disorder comprising administering to a subject in need thereof an effective amount of a Pyrimido[5,4-c]quinoline-2,4-diamine Derivative.

The invention further provides methods for modulating an activity of PDK-1 comprising administering to a subject in need thereof an effective amount of a Pyrimido[5,4-c]quinoline-2,4-diamine Derivative, wherein it is known that PDK-1 activity is related to a disease or condition. In one embodiment, the disease or condition is cancer.

The invention provides a process for the preparation of a compound of Formula (I) comprising reacting a compound of Formula (II)

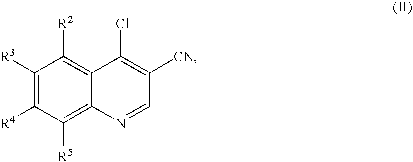

wherein R², R³, R⁴, and R⁵ are as described herein,

with a compound of Formula (III)

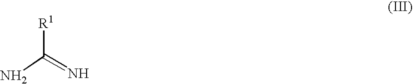

wherein R¹ is as described herein.

The details of the invention are set forth in the accompanying description below.

All patents and publications cited in this specification are incorporated by reference in their entirety.

### 5. DETAILED DESCRIPTION OF THE INVENTION

### 5.1. Definitions and Abbreviations

The following definitions are used in connection with the Pyrimido[5,4-c]quinoline-2,4-diamine Derivatives:

As used herein, unless specifically indicated otherwise, the conjunction “or” is used in the inclusive sense of “and/or” and not the exclusive sense of “either/or.”

“C₁-C₄ alkyl” refers to a straight or branched chain saturated hydrocarbon containing 1-4 carbon atoms. Representative C₁-C₄ alkyl groups include methyl, ethyl, propyl, isopropyl, butyl, tert-butyl, sec-butyl, and isobutyl. In one embodiment, the C₁-C₄ alkyl group is substituted with one or more of the following groups: -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

“C₁-C₆ alkyl” refers to a straight or branched chain saturated hydrocarbon containing 1-6 carbon atoms. Representative C₁-C₆ alkyl groups include, but are not limited to, methyl, ethyl, propyl, isopropyl, butyl, tert-butyl, sec-butyl, isobutyl, pentyl, isopentyl, neopentyl, hexyl, isohexyl and neohexyl. In one embodiment, the C₁-C₆ alkyl group is substituted with one or more of the following groups: -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

“C₂-C₆ alkenyl” refers to a straight or branched chain unsaturated hydrocarbon containing 2-6 carbon atoms and at least one double bond. Representative C₂-C₆ alkenyl groups include, but are not limited to, ethylene, propylene, 1-butylene, 2-butylene, isobutylene, sec-butylene, 1-pentene, 2-pentene, isopentene, 1-hexene, 2-hexene, 3-hexene and isohexene. In one embodiment, the C₂-C₆ alkenyl group is substituted with one or more of the following groups: -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

“C₂-C₆ alkynyl” refers to a straight or branched chain unsaturated hydrocarbon containing 2-6 carbon atoms and at least one triple bond. Representative C₂-C₆ alkynyl groups include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne and isohexyne. In one embodiment, the C₂-C₆ alkynyl group is substituted with one or more of the following groups: -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

“C₁-C₆ alkylene” refers to a straight or branched chain saturated hydrocarbon containing 1-6 carbon atoms, wherein two of the hydrocarbon's hydrogen atoms have been replaced by a single a bond. Representative C₁-C₆ alkylene groups include, but are not limited to, methylene, ethylene, n-propylene, isopropylene, n-butylene, isobutylene, n-pentylene, isopentylene, and n-hexylene.

“C₁-C₄ alkylene” refers to a straight or branched chain saturated hydrocarbon containing 1-4 carbon atoms, wherein two of the hydrocarbon's hydrogen atoms have been replaced by a single a bond. Representative C₁-C₄ alkylene groups include methylene, ethylene, n-propylene, isopropylene, n-butylene, and isobutylene.

A “phenylene” is a substituted or unsubstituted phenyl group, wherein two of the phenyl group's hydrogen atoms have been replaced by a single a bond, e.g., 1,2- or 1,3- or 1,4-phenylene.

“Halo” refers to —F, —Cl, —Br or —I.

“Halo-substituted C₁-C₄ alkyl” refers to a C₁-C₄ alkyl group, as defined above, wherein one or more of the C₁-C₄ alkyl group's hydrogen atoms has been replaced with —F, —Cl, —Br or —I. Representative examples of a halo-substituted C₁-C₄ alkyl include, but are not limited to, —CH₂F, —CCl₃, —CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH(Br)CH₃, —CH₂CH(Cl)CH₂CH₃, —CH(F)CH₂CH₃ and —C(CH₃)₂(CH₂Cl). In one embodiment, one or more is one to three.

A “C₃-C₈ monocyclic cycloalkyl” is a non-aromatic, saturated hydrocarbon ring containing 3-8 carbon atoms. Representative C₃-C₈ monocyclic cycloalkyl groups include, but are not limited to, cyclopropyl, cyclobutyl, cyclopentyl, cyclohexyl, cycloheptyl and cyclooctyl.

In one embodiment, the C₃-C₈ monocyclic cycloalkyl group is unsubstituted or substituted with one or more of the following groups: -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

A “C₃-C₈ monocyclic cycloalkenyl” is a non-aromatic hydrocarbon ring containing 3-8 carbon atoms and having at least one endocyclic double bond. Representative C₃-C₈ monocyclic cycloalkenyl groups include, but are not limited to, cyclopropenyl, cyclobutenyl, 1,3-cyclobutadienyl, cyclopentenyl, 1,3-cyclopentadienyl, cyclohexenyl, 1,3-cyclohexadienyl, cycloheptenyl, 1,3-cycloheptadienyl, 1,4-cycloheptadienyl, -1,3,5-cycloheptatrienyl, cyclooctenyl, 1,3-cyclooctadienyl, 1,4-cyclooctadienyl, -1,3,5-cyclooctatrienyl.

The term “3- to 7-membered non-aromatic monocyclic heterocycle” refers to a 3-7 membered saturated or partially unsaturated ring having 1 to 3 heteroatoms independently selected from N, O and S, where any carbon ring atom may form a carbonyl or thiocarbonyl group, and where any ring nitrogen or sulfur atom may be oxidised. Included in this class are (1) a 3- or 4-membered non-aromatic monocyclic cycloalkyl in which one of the ring carbon atoms has been replaced with a N, O or S atom; or (2) a 5-, 6-, or 7-membered non-aromatic monocyclic cycloalkyl in which 1 to 3 of the ring carbon atoms has been independently replaced with an N, O or S atom. In one embodiment, the 3- to 7-membered non-aromatic monocyclic heterocycle is a 4- to 7-membered non-aromatic monocyclic heterocycle. In one embodiment, a carbon atom of a 4- to 7-membered non-aromatic monocyclic heterocycle is replaced with a carbonyl group. In one embodiment, a carbon atom of a 3- to 7-membered non-aromatic monocyclic heterocycle is replaced with a carbonyl group. In another embodiment, a carbon atom of a 3- to 7-membered non-aromatic monocyclic heterocycle is replaced with a thiocarbonyl group. A 3- to 7-membered non-aromatic monocyclic heterocycle can be attached via a ring nitrogen or ring carbon atom. Representative examples of a 3- to 7-membered non-aromatic monocyclic heterocycle group include, but are not limited to azepanyl, aziridinyl, 1,3-dioxolanyl, 1,4-dioxolanyl, imidazolidinyl, imidazolidin-2-one-yl, imidazolinyl, morpholinyl, piperazinyl, N-methylpiperazinyl, piperidinyl, N-methylpiperidinyl, pyranyl, pyrazolidinyl, pyrazolinyl, pyrrolidinonyl, pyrrolidinyl, N-methylpyrrolidinyl, N-benzylpyrrolidinyl, pyrrolinyl, tetrahydrofuranyl, tetrahydropyranyl, thiomorpholinyl, thiomorpholinyl-5-oxide, thiomorpholinyl-S,S-dioxide and trithianyl.

In one embodiment, the 3- to 7-membered non-aromatic monocyclic heterocycle group is independently substituted with one or more of the following groups: —R¹³, —OR¹³, —R¹⁴, 4-(2,3-dihydro-indol-1-yl), 4-(1,3-dihydro-isoindol-2-yl), —NH-benzoyl, wherein R¹³ and R¹⁴ are as defined above for the compounds of formula (I). In another embodiment, the 3- to 7-membered non-aromatic monocyclic heterocycle group is substituted on a single saturated C atom not adjacent to a N atom with —O(CH₂)₂O— or —(CH₂)₃O—. In one embodiment, one or more is one to three.

The term “3- to 7-membered monocyclic heterocycle” refers to a monocyclic 3- to 7-membered aromatic or non-aromatic monocyclic cycloalkyl in which 1-4 of the ring carbon atoms have been independently replaced with a N, O or S atom. The 3- to 7-membered monocyclic heterocycles can be attached via a nitrogen, sulfur, or carbon atom. Representative examples of a 3- to 7-membered monocyclic heterocycle group include, but are not limited to, piperidinyl, piperazinyl, pyrrolyl, piperidonyl, oxazinyl, thiazinyl, diazinyl, triazinyl, tetrazinyl, imidazolyl, tetrazolyl, pyrrolidinyl, isoxazolyl, pyridinyl, oxazolyl, thiazolyl, pyrazolyl, triazolyl, pyrimidinyl, morpholinyl, furuzanyl, pyrrolinyl, imidazolinyl, imidazolidinyl, pyrazolinyl, isothiazolyl, oxadiazolyl, thiadiazolyl, pyridazinyl, pyrazinyl, pyrazolidinyl, thiomorpholinyl, tetrahydrofuranyl, dihydrofuranyl, pyranyl, dihydropyranyl, tetrahydropyranyl, thiopyranyl, dihydrothiopyranyl, tetrahydrothiopyranyl, dioxanyl, dithianyl, trithianyl, dioxolanyl, furanyl, and thiophenyl. In one embodiment, the 3- to 7-membered monocyclic heterocycle is a non-aromatic 3- to 7-membered monocyclic heterocycle. In another embodiment, the 3- to 7-membered monocyclic heterocycle is saturated or partially saturated.

The term “5- or 6-membered aromatic monocyclic heterocycle” refers to a 5- or 6-membered aromatic monocyclic cycloalkyl in which from 1 to 4 of the ring carbon atoms has been replaced with an N, O or S atom. In one embodiment, the 5- or 6-membered aromatic monocyclic heterocycle is attached via a ring carbon atom. Representative examples of a 5- or 6-membered aromatic monocyclic heterocycle group include, but are not limited to furanyl, imidazolyl, isothiazolyl, isoxazolyl, oxazolyl, oxadiazolyl, pyrimidinyl, pyrazolyl, pyrazinyl, pyridazinyl, pyridinyl, pyrrolyl, thiazolyl, thiadiazolyl, thiophenyl, triazinyl, and triazolyl.

In one embodiment, the 5- or 6-membered aromatic monocyclic heterocycle group is independently substituted with one or more of the following groups: —R¹³, —OR¹³, —R¹⁴, 4-(2,3-dihydro-indol-1-yl), 4-(1,3-dihydro-isoindol-2-yl), —NH-benzoyl, wherein R¹³ and R¹⁴ are as defined above for the compounds of formula (I). In one embodiment, one or more is one to three.

The term “8- to 12-membered bicyclic heterocycle” refers to a bicyclic 8- to 12-membered aromatic or non-aromatic bicyclic cycloalkyl in which one or both of the of the rings of the bicyclic ring system have 1-4 of its ring carbon atoms independently replaced with a N, O or S atom. Included in this class are 3- to 7-membered monocyclic heterocycles that are fused to a benzene ring. A non-aromatic ring of an 8- to 12-membered bicyclic heterocycle is attached via a ring nitrogen or ring carbon atom. An aromatic 8- to 12-membered bicyclic heterocycle is attached via a ring carbon atom. Examples of 8- to 12-membered bicyclic heterocycles include, but are not limited to, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, cinnolinyl, decahydroquinolinyl, 1H-indazolyl, indolenyl, indolinyl, indolizinyl, indolyl, isobenzofuranyl, isoindazolyl, isoindolyl, isoindolinyl, isoquinolinyl, naphthyridinyl, octahydroisoquinolinyl, phthalazinyl, pteridinyl, purinyl, quinoxalinyl, tetrahydroisoquinolinyl, tetrahydroquinolinyl, and xanthenyl.

In one embodiment, each ring of the 8- to 12-membered bicyclic heterocycle group is independently substituted with one or more of the following groups: —R¹³, —OR¹³, —R¹⁴, 4-(2,3-dihydro-indol-1-yl), 4-(1,3-dihydro-isoindol-2-yl), —NH-benzoyl, wherein R¹³ and R¹⁴ are as defined above for the compounds of formula (I). In another embodiment, the −8- to 12-membered bicyclic heterocycle group can be substituted on a single saturated C atom not adjacent to a N atom with —O(CH₂)₂O— or —(CH₂)₃O—. In one embodiment, a carbon atom of a −8- to 12-membered bicyclic heterocycle group is replaced with a carbonyl group. In another embodiment, a carbon atom of a −8- to 12-membered bicyclic heterocycle group is replaced with a thiocarbonyl group. In one embodiment, one or more is one to three.

A “subject” is a mammal, e.g., a human, mouse, rat, guinea pig, dog, cat, horse, cow, pig, or non-human primate, such as a monkey, chimpanzee, baboon. In one embodiment, the monkey is a rhesus. In one embodiment, a subject is a human.

The phrase “pharmaceutically acceptable salt,” as used herein, is a salt formed from an acid and a basic nitrogen group of one of the Pyrimido[5,4-c]quinoline-2,4-diamine Derivatives. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, besylate, mesylate, camphor sulfonate, and pamoate (i.e., 1,1′-methylene-bis-(2-OH-3-naphthoate)) salts.

The term “pharmaceutically acceptable salt” also refers to a salt prepared from a Pyrimido[5,4-c]quinoline-2,4-diamine Derivative having an acidic functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, tris-(hydroxymethyl)methylamine, or 2-hydroxy-tert-butylamine, or N,N-di-lower alkyl-N-(hydroxy lower alkyl)-amines, such as N,N-dimethyl-N-(2-hydroxyethyl)amine or tri-(2-hydroxyethyl)amine; N-methyl-D-glucamine; and amino acids such as arginine, lysine, and the like, wherein ‘lower’ denotes 1 to 6 carbon atoms. A Pyrimido[5,4-c]quinoline-2,4-diamine Derivative can also exist in a form of a hydrate.

An “effective amount” when used in connection with a Pyrimido[5,4-c]quinoline-2,4-diamine Derivative is an amount effective for treating or preventing a proliferative disorder.

An “effective amount” when used in connection with another anticancer agent is an amount that is effective for treating or preventing cancer alone or in combination with a Pyrimido[5,4-c]quinoline-2,4-diamine Derivative.

“In combination with” includes administration within the same composition and within separate compositions. In the latter instance, the anticancer agent is administered during a time when the Pyrimido[5,4-c]quinoline-2,4-diamine Derivative exerts its prophylactic or therapeutic effect, or vice versa.

Some chemical structures herein are depicted using bold and dashed lines to represent chemical bonds. These bold and dashed lines depict absolute stereochemistry. Unless otherwise indicated, a chiral center without any indication of stereochemistry represents (R) isomer, (S) isomer, or any mixture of the two.

Illustrative examples of the Pyrimido[5,4-c]quinoline-2,4-diamine Derivative are described herein using both chemical structures and chemical names. It is to be understood that if a discrepancy exists between a chemical structure and its corresponding chemical name, the chemical structure predominates.

The compounds of this invention may contain one or more asymmetric carbon atoms. In such cases, the compounds of this invention include the individual diasteromers, the racemates, and the individual R and S entantiomers thereof. Some of the compounds of this invention may contain one or more double bonds. In such cases, the compounds of this invention include each of the possible configurational isomers as well as mixtures of these isomers. Some of the compounds of this invention may exist as separate tautomers. In such cases, the compounds of this invention include each tautomer and mixtures of these tautomers.

When a compound of this invention has a moiety that contains a heterocyclic ring, either mono, bicyclic, or tricyclic, such heterocyclic ring does not contain O—O, S—S, or S—O bonds in the ring.

The following abbreviations are used herein and have the indicated definitions: DMF is N,N-dimethylformamide, Me is methyl, Me is methyl, MS is mass spectrometry, THF is tetrahydrofuran, His is histidine, NTA is nitrilotriacetic acid, and HRP is horseradish peroxidase.

### 5.2. The Pyrimido[5,4-c]quinoline-2,4-diamine Derivatives of Formula (I)

The invention provides Pyrimido[5,4-c]quinoline-2,4-diamine Derivatives according to Formula (I):

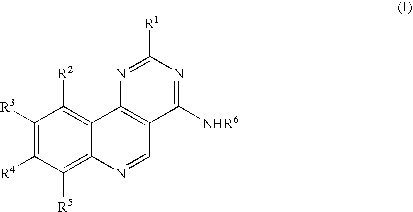

and pharmaceutically acceptable salts thereof,
wherein
R¹, R², R³, R⁴, R⁵ and R⁶, are as defined above for the compounds of formula (I).

In one embodiment, R², R⁵ and/or R⁶ is —H.

In another embodiment, one or both of R³ and R⁴ is —O—C₁-C₆ alkyl.

In yet another embodiment, R¹ is —NH₂.

In a further embodiment, R⁴ is —O—C₁-C₆ alkylene-O—C₁-C₆ alkyl.

In one embodiment, R¹ is —N(C₁-C₆ alkyl)₂.

In another embodiment, R¹ is —NR¹²R¹².

In yet another embodiment, one or more R¹² is —H.

In a further embodiment, one or more R¹² is —C₃-C₈ monocyclic cycloalkyl.

In one embodiment, one or more R¹² is -benzyl.

In another embodiment, R¹ is morpholino.

In one embodiment, the compounds of Formula (I) have the Formula (Ia):

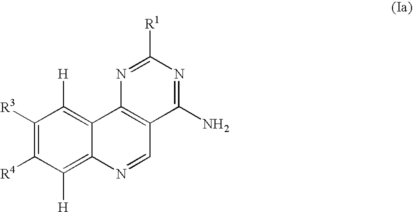

wherein R¹, R³ and R⁴ and as defined above for Formula (I).

In one embodiment for the compounds of Formula (Ia), R³ and R⁴ are —O—C₁-C₆ alkyl.

In another embodiment for the compounds of Formula (Ia), R³ and R⁴ are —O—C₁-C₆ alkyl.

In yet another embodiment for the compounds of Formula (Ia), R³ and R⁴ are —O—C₁-C₆ alkyl and R¹ is —NH₂.

In a further embodiment for the compounds of Formula (Ia), R³ and R⁴ are —O—C₁-C₆ alkyl, and R¹ is —N(C₁-C₆ alkyl)₂.

In one embodiment for the compounds of Formula (Ia), R³ and R⁴ are —O—C₁-C₆ alkyl, and R¹ is —N(R¹²)₂.

In another embodiment for the compounds of Formula (Ia), R³ and R⁴ are —O—C₁-C₆ alkyl, R¹ is —N(R¹²)₂, and one or more R¹² is —H.

In yet another embodiment for the compounds of Formula (Ia), R³ and R⁴ are —O—C₁-C₆ alkyl, R¹ is —N(R¹²)₂, and one or more R¹² is —C₃-C₈ monocyclic cycloalkyl.

In a further embodiment for the compounds of Formula (Ia), R³ and R⁴ are —O—C₁-C₆ alkyl, R¹ is —N(R¹²)₂, and one or more R¹² is -benzyl.

In one embodiment for the compounds of Formula (Ia), R¹ is —NH₂.

In another embodiment for the compounds of Formula (Ia), R¹ is —N(CH₃)₂.

In yet another embodiment for the compounds of Formula (Ia), R¹ is —NH(CH₂CH₃).

In various embodiments for the compounds of Formula (Ia), R¹ is

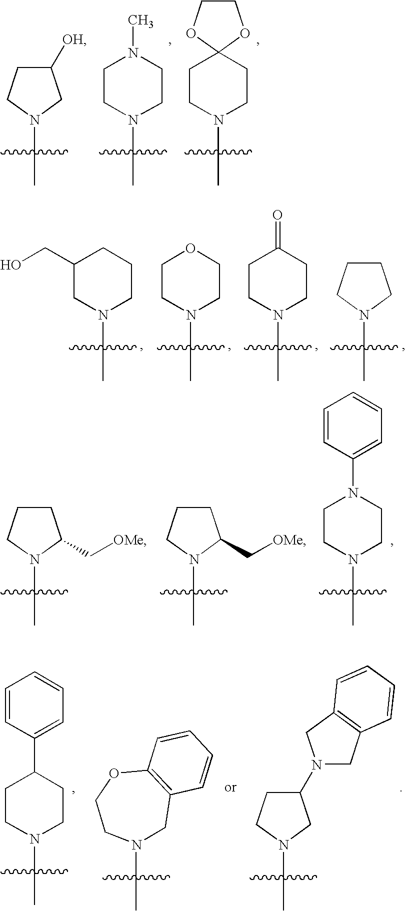

Illustrative examples of the Pyrimido[5,4-c]quinoline-2,4-diamine Derivatives include the compounds of Formula (Ia) as set forth below:

| Compound | R¹ | R³ | R⁴ |
|---|---|---|---|
| Ia-1 | —NH₂ | —OCH₃ | —OCH₃ |
| Ia-2 | —NH₂ | —O—CH₂—CH₃ | —O—CH₂—CH₃ |
| Ia-3 | —NH₂ | —OCH₃ | —F |
| Ia-4 | 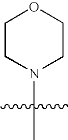 | —O—CH₂—CH₃ | —O—CH₂—CH₃ |
| Ia-5 | 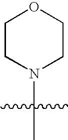 | —OCH₃ | —O—(CH₂)₂—O—CH₃ |
| Ia-6 | 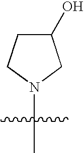 | —OCH₃ | —O—(CH₂)₂—O—CH₃ |
| Ia-7 | 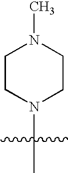 | —OCH₃ | —O—(CH₂)₂—O—CH₃ |
| Ia-8 | —N(CH₃)₂ | —OCH₃ | —O—(CH₂)₂—O—CH₃ |
| Ia-9 | —NH—CH₂—CH₃ | —OCH₃ | —O—(CH₂)₂—O—CH₃ |
| Ia-10 | 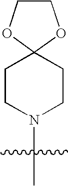 | —OCH₃ | —O—(CH₂)₂—O—CH₃ |
| Ia-11 | 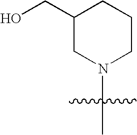 | —OCH₃ | —O—(CH₂)₂—O—CH₃ |
| Ia-12 | —NH-(p-chloro- | —OCH₃ | —O—(CH₂)₂—O—CH₃ |
| | phenyl) | | |
| Ia-13 | 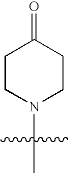 | —OCH₃ | —O—(CH₂)₂—O—CH₃ |
| Ia-14 | 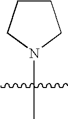 | —OCH₃ | —O—(CH₂)₂—O—CH₃ |
| Ia-15 | 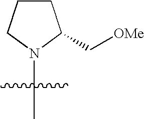 | —OCH₃ | —O—(CH₂)₂—O—CH₃ |
| Ia-16 | 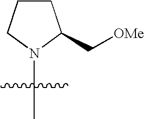 | —OCH₃ | —O—(CH₂)₂—O—CH₃ |
| Ia- 17 | —NH-cyclohexyl | —OCH₃ | —O—(CH₂)₂—O—CH₃ |
| Ia- 18 | 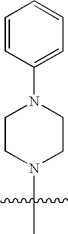 | —OCH₃ | —O—(CH₂)₂—O—CH₃ |
| Ia-19 | 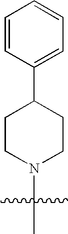 | —OCH₃ | —O—(CH₂)₂—O—CH₃ |
| Ia-20 | 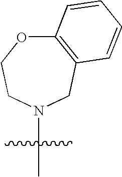 | —OCH₃ | —O—(CH₂)₂—O—CH₃ |
| Ia-21 | —NH-benzyl | —OCH₃ | —O—(CH₂)₂—O—CH₃ |
| Ia-22 | 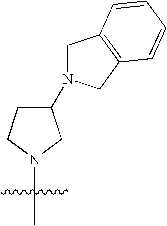 | —OCH₃ | —O—(CH₂)₂—O—CH₃ |
| Ia-23 | —NH-(m-chloro- | —OCH₃ | —O—(CH₂)₂—O—CH₃ |
| | phenyl) | | |

and pharmaceutically acceptable salts thereof.

In some embodiments, the Pyrimido[5,4-c]quinoline-2,4-diamine Derivatives of Formula (I) and/or (Ia) are:
8,9-dimethoxypyrimido[5,4-c]quinoline-2,4-diamine;
8,9-diethoxypyrimido[5,4-c]quinoline-2,4-diamine;
8-fluoro-9-methoxypyrimido[5,4-c]quinoline-2,4-diamine;
8,9-diethoxy-2-morpholin-4-ylpyrimido[5,4-c]quinolin-4-amine;
9-methoxy-8-(2-methoxyethoxy)-2-morpholin-4-ylpyrimido[5,4-c]quinolin-4-amine;
1-[4-amino-9-methoxy-8-(2-methoxyethoxy)pyrimido[5,4-c]quinolin-2-yl]pyrrolidin-3-ol;
9-methoxy-8-(2-methoxyethoxy)-2-(4-methylpiperazin-1-yl)pyrimido[5,4-c]quinolin-4-amine;
9-methoxy-8-(2-methoxyethoxy)-N²,N²-dimethylpyrimido[5,4-c]quinoline-2,4-diamine;
N²-ethyl-9-methoxy-8-(2-methoxyethoxy)pyrimido[5,4-c]quinoline-2,4-diamine;
2-(1,4-dioxa-8-azaspiro[4.5]dec-8-yl)-9-methoxy-8-(2-methoxyethoxy)pyrimido[5,4-c]quinolin-4-amine;
N²-(4-chlorophenyl)-9-methoxy-8-(2-methoxyethoxy)pyrimido[5,4-c]quinoline-2,4-diamine;
{1-[4-amino-9-methoxy-8-(2-methoxyethoxy)pyrimido[5,4-c]quinolin-2-yl]piperidin-3-yl}methanol;
1-[4-amino-9-methoxy-8-(2-methoxyethoxy)pyrimido[5,4-c]quinolin-2-yl]piperidin-4-one;
9-methoxy-8-(2-methoxyethoxy)-2-pyrrolidin-1-ylpyrimido[5,4-c]quinolin-4-amine;
9-methoxy-8-(2-methoxyethoxy)-2-[(2R)-2-(methoxymethyl)pyrrolidin-1-yl]pyrimido[5,4-c]quinolin-4-amine;
9-methoxy-8-(2-methoxyethoxy)-2-[(2S)-2-(methoxymethyl)pyrrolidin-1-yl]pyrimido[5,4-c]quinolin-4-amine;
N²-cyclohexyl-9-methoxy-8-(2-methoxyethoxy)pyrimido[5,4-c]quinoline-2,4-diamine;
9-methoxy-8-(2-methoxyethoxy)-2-(4-phenylpiperazin-1-yl)pyrimido[5,4-c]quinolin-4-amine;
9-methoxy-8-(2-methoxyethoxy)-2-(4-phenylpiperidin-1-yl)pyrimido[5,4-c]quinolin-4-amine;
2-(2,3-dihydro-1,4-benzoxazepin-4(5H)-yl)-9-methoxy-8-(2-methoxyethoxy)pyrimido[5,4-c]quinolin-4-amine;
N²-(3-chlorophenyl)-9-methoxy-8-(2-methoxyethoxy)pyrimido[5,4-c]quinoline-2,4-diamine;
2-[3-(1,3-dihydro-2H-isoindol-2-yl)pyrrolidin-1-yl]-9-methoxy-8-(2-methoxyethoxy)pyrimido[5,4-c]quinolin-4-amine;
N²-benzyl-9-methoxy-8-(2-methoxyethoxy)pyrimido[5,4-c]quinoline-2,4-diamine,
   or a pharmaceutically acceptable salt thereof.

### 5.3. Methods for Making the Pyrimido[5,4-c]quinoline-2,4-diamine Derivatives

Examples of synthetic pathways that are useful for making Pyrimido[5,4-c]quinoline-2,4-diamine Derivatives are set forth in the Examples below and generalized in Schemes 1-3.

The compounds of this invention represented by formula 3 can be prepared as shown in Scheme 1.

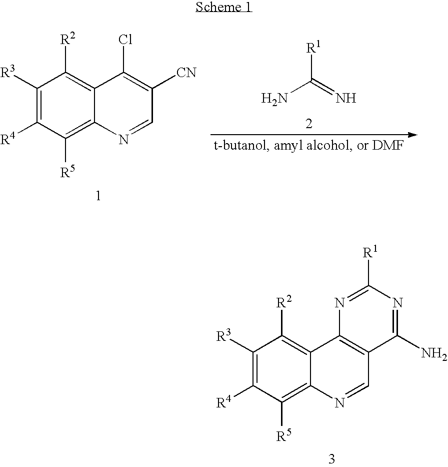

The reaction of a 4-chloro-3-cyanoquinoline compound of formula 1 with an acid salt of a guanidine compound 2, in an inert solvent, e.g., DMF or a tertiary alcohol, e.g., t-butanol or amyl alcohol, in the presence of a base, such as a tertiary amine or an in inorganic base (e.g., potassium carbonate, sodium acetate) at elevated temperature, yields the pyrimido[5,4-c]quinoline-2,4-diamine compound of formula 3, wherein R¹, R², R³, R⁴ and R⁵ are as defined above for the compounds of Formula (I). The guanidine acid salts of formula 2 are commercially available or can be prepared by well known methods. The primary amine of pyrimido[5,4-c]quinoline-2,4-diamine compound of formula 3, can be alkylated or oxidized using methods known in the art (see e.g., March, Advanced Organic Chemistry: Reactions, Mechanisms and Structure, Fourth Edition, John Wiley and Sons, 1992, incorporated by reference herein in its entirety) to attach R⁶, wherein R⁶ is as defined above for the compounds of Formula (I). Alternatively, R⁶ can be attached to the primary amine of the pyrimido[5,4-c]quinoline-2,4-diamine compound of formula 3 by substituting the amino group of the compound of formula 2 with R⁶.

For example, as shown in Scheme 2, the reaction of an amine of formula 4, wherein hydrogen is attached to a nitrogen atom of R¹, as defined above for the compounds of Formula (I), with a thiourea reagent 5 in water or a water/alcohol mixture at 90-100° C., yields a guanidine acid salt of formula 6.

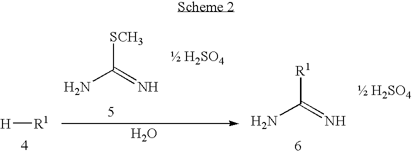

The methods used to prepare the 4-chloro-3-cyanoquinoline compounds of formula 1 are described in detail in the following patents and patent applications: WO 98/43960, U.S. Pat. No. 6,288,082, U.S. Pat. No. 6,002,008, WO 00/18761 and WO 00/18740, all of which are incorporated by reference herein in their entirety. A typical method is shown below in Scheme 3.

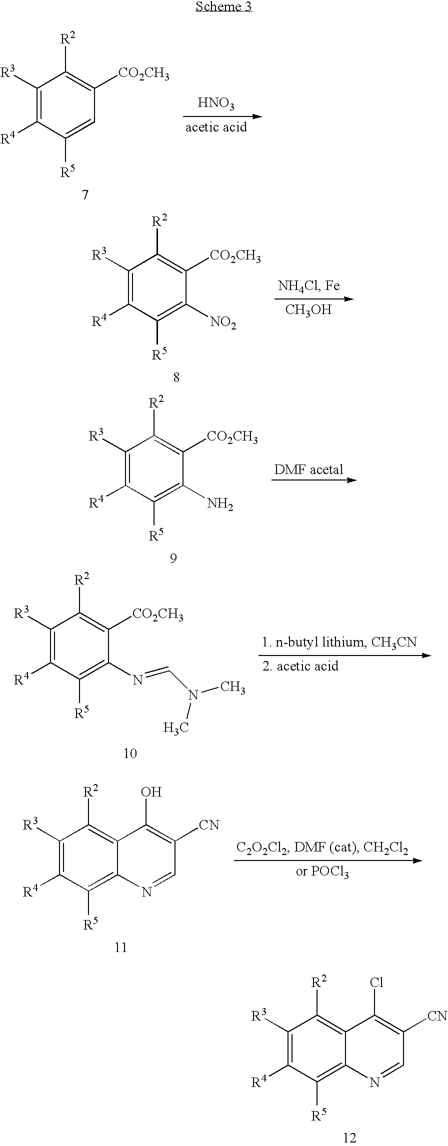

A benzoic acid ester of formula 7, wherein R², R³, R⁴ and R⁵ are as defined above for the compounds of Formula (I) can be nitrated to provide a nitrate of formula 8. Where necessary, substituents on the benzoic ester of formula 7 can be protected with a suitable protecting group compatible with the subsequent reactions (see e.g., T. W. Green and P. G. M. Wuts, Protective Groups in Organic Synthesis, Wiley-Interscience, New York, 1999, incorporated herein by reference in its entirety). If regioisomers are obtained in the nitration step, they can be separated by various methods, such as chromatography and fractional recrystallization. The nitro derivative of formula 8 can be reduced, for example, using a refluxing mixture of ammonium chloride and iron in methanol or similar solvent, to yield an amine of formula 9. The amino group of the compound of formula 9 can be converted to an amidine derivative of formula 10, e.g., with a DMF-acetal. The reaction of the amidine compound of formula 10 with an excess of the lithium anion of acetonitrile at low temperature, which can be produced by the reaction of acetonitrile with n-butyl lithium at low temperature, followed by warming and treatment with acetic acid results in the formation of a 3-cyano-4-hydroxy-quinoline compound of formula 11. The 4-chloro-3-cyanoquinoline compound of formula 12, useful in preparation of the compounds described herein, can obtained, for example, by reaction of a cyanoquinoline compound of formula 11 with oxalyl chloride in refluxing methylene chloride in the presence of a catalytic amount of DMF or, alternatively, by heating the cyanoquinoline compound 11 with phosphorous oxychloride.

Compounds made using the methods provided above in Schemes 1-3 can be derivatized using methods known to one skilled in the art of organic synthesis in order to provide the entire scope of the Pyrimido[5,4-c]quinoline-2,4-diamine Derivatives of formula (I).

### 5.4. Methods of Using the Pyrimido[5,4-c]quinoline-2,4-diamine Derivatives

In accordance with the invention, the Pyrimido[5,4-c]quinoline-2,4-diamine Derivatives are useful for treatment or prevention of a proliferative disorder. Furthermore, Pyrimido[5,4-c]quinoline-2,4-diamine Derivatives are useful for modulation of activity of PDK-1.

### 5.4.1. Methods for Treating or Preventing a Proliferative Disorder

A proliferative disorder can be treated or prevented by administration of an effective amount of a Pyrimido[5,4-c]quinoline-2,4-diamine Derivative.

Proliferative disorders that can be treated or prevented by administering an effective amount of a Pyrimido[5,4-c]quinoline-2,4-diamine Derivative include, but are not limited to, cancer, uterine fibroids, benign prostatic hyperplasia, familial adenomatosis polyposis, neurofibromatosis, atherosclerosis, pulmonary fibrosis, arthritis, psoriasis, glomerulonephritis, restenosis following angioplasty or vascular surgery, hypertrophic scar formation, an inflammatory bowel disease, transplantation rejection, endotoxic shock, a fungal infection, a defective apoptosis-associated condition, or a proliferative disease that is dependent on PDK-1 activity.

In one embodiment, the proliferative disorder is cancer.

In another embodiment, the proliferative disorder is a proliferative disorder that is dependent on PDK-1 activity.

### 5.4.2. Methods for Treating or Preventing Cancer

The Pyrimido[5,4-c]quinoline-2,4-diamine Derivatives are useful for treating or preventing cancer.

The invention provides methods for treating or preventing cancer, comprising administering to a subject in need of such treatment or prevention an effective amount of a Pyrimido[5,4-c]quinoline-2,4-diamine Derivative.

Examples of cancers treatable or preventable using the Pyrimido[5,4-c]quinoline-2,4-diamine Derivatives include, but are not limited to, a cancer which expresses PDK-1, the cancers disclosed below in Table 1 and metastases thereof.

TABLE 1

Solid tumors, including but not limited to:
fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon cancer
colorectal cancer
kidney cancer
pancreatic cancer
bone cancer
breast cancer
ovarian cancer
prostate cancer
esophageal cancer
stomach cancer
oral cancer
nasal cancer
throat cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular cancer
small cell lung carcinoma
bladder carcinoma
lung cancer
epithelial carcinoma
skin cancer
melanoma
neuroblastoma
retinoblastoma
Blood-borne cancers, including but not limited to:
acute lymphoblastic leukemia
acute lymphoblastic B-cell leukemia
acute lymphoblastic T-cell leukemia
acute myeloblastic leukemia
acute promyelocytic leukemia
acute monoblastic leukemia
acute erythroleukemic leukemia
acute megakaryoblastic leukemia
acute myelomonocytic leukemia
acute nonlymphocyctic leukemia
acute undifferentiated leukemia
chronic myelocytic leukemia
chronic lymphocytic leukemia
hairy cell leukemia
multiple myeloma
Lymphomas, including but not limited to:
Hodgkin's disease
non-Hodgkin's Lymphomas:
Multiple myeloma
Waldenström's macroglobulinemia
Heavy chain disease
Polycythemia vera
CNS and brain cancers including, but not limited to:
glioma
pilocytic astrocytoma
astrocytoma
anaplastic astrocytoma
glioblastoma multiforme
medulloblastoma
craniopharyngioma
ependymoma
pinealoma
hemangioblastoma
acoustic neuroma
oligodendroglioma
meningioma
vestibular schwannoma
adenoma
metastatic brain tumor
meningioma
spinal tumor
medulloblastoma

In one embodiment, the cancer is lung cancer, breast cancer, colorectal cancer, prostate cancer, a leukemia, a lymphoma, a skin cancer, a brain cancer, a cancer of the central nervous system, ovarian cancer, uterine cancer, stomach cancer, pancreatic cancer, esophageal cancer, kidney cancer, liver cancer, or a head and neck cancer.

In another embodiment, the cancer is metastatic cancer.

In yet another embodiment, the cancer is a cancer which expresses PDK-1.

In still another embodiment, the subject has previously undergone or is presently undergoing treatment for cancer. Such previous treatments include, but are not limited to, prior chemotherapy, radiation therapy, surgery or immunotherapy, such as cancer vaccines.

The Pyrimido[5,4-c]quinoline-2,4-diamine Derivatives are also useful for the treatment or prevention of a cancer caused by a virus. Such viruses include human papilloma virus, which can lead to cervical cancer (see, e.g., Hernandez-Avila et al., Archives of Medical Research (1997) 28:265-271); Epstein-Barr virus (EBV), which can lead to lymphoma (see, e.g., Herrmann et al., J Pathol (2003) 199(2):140-5); hepatitis B or C virus, which can lead to liver carcinoma (see, e.g., El-Serag, J Clin Gastroenterol (2002) 35(5 Suppl 2):S72-8); human T cell leukemia virus (HTLV)-I, which can lead to T-cell leukemia (see e.g., Mortreux et al., Leukemia (2003) 17(1):26-38); human herpesvirus-8 infection, which can lead to Kaposi's sarcoma (see, e.g., Kadow et al., Curr Opin Investig Drugs (2002) 3(11): 1574-9); and Human Immune deficiency Virus (HIV) infection, which can lead to cancer as a consequence of immunodeficiency (see, e.g., Dal Maso et al., Lancet Oncol (2003) 4(2):110-9).

The Pyrimido[5,4-c]quinoline-2,4-diamine Derivatives can also be administered to prevent the progression of a cancer, including but not limited to the cancers listed in Table 1. Such prophylactic use includes that in which non-neoplastic cell growth consisting of hyperplasia, metaplasia, or most particularly, dysplasia has occurred.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a subject, can indicate the desirability of prophylactic/therapeutic administration of the Pyrimido[5,4-c]quinoline-2,4-diamine Derviatives. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, are treatable or preventable according to the present methods.

In another embodiment, fibrocystic disease (cystic hyperplasia, mammary dysplasia, particularly adenosis (benign epithelial hyperplasia)) are treatable or preventable according to the present methods.

In other embodiments, a subject that exhibits one or more of the following predisposing factors for malignancy can be administered an amount of a Pyrimido[5,4-c]quinoline-2,4-diamine Derivative which is effective to treat or prevent cancer: a chromosomal translocation associated with a malignancy (e.g., the Philadelphia chromosome for chronic myelogenous leukemia, t(14;18) for follicular lymphoma); familial polyposis or Gardner's syndrome; benign monoclonal gammopathy; a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; and exposure to carcinogens (e.g., smoking, second-hand smoke exposure, and inhalation of or contacting with certain chemicals).

### 5.4.3. Combination Chemotherapy for the Treatment of Cancer

In one embodiment, the present methods for treating cancer or preventing cancer further comprise administering another anticancer agent.

In one embodiment, the present invention provides methods for treating or preventing cancer in a subject, the method comprising the administration of an effective amount of: (i) a Pyrimido[5,4-c]quinoline-2,4-diamine Derivative and (ii) another anticancer agent.

In one embodiment, (i) a Pyrimido[5,4-c]quinoline-2,4-diamine Derivative and (ii) another anticancer agent are administered in doses commonly employed when such agents are used as monotherapy for the treatment of cancer.

In another embodiment, (i) a Pyrimido[5,4-c]quinoline-2,4-diamine Derivative and (ii) another anticancer agent act synergistically and are administered in doses that are less than the doses commonly employed when such agents are used as monotherapy for the treatment of cancer.

The dosage of the (i) a Pyrimido[5,4-c]quinoline-2,4-diamine Derivative, and (ii) another anticancer agent administered as well as the dosing schedule can depend on various parameters, including, but not limited to, the cancer being treated, the subject's general health, and the administering physician's discretion.

A Pyrimido[5,4-c]quinoline-2,4-diamine Derivative can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the other anticancer agent to a subject in need thereof. In various embodiments, i) a Pyrimido[5,4-c]quinoline-2,4-diamine Derivative, and (ii) another anticancer agent are administered 1 minute apart, 10 minutes apart, 30 minutes apart, less than 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart, or no more than 48 hours apart. In one embodiment, i) a Pyrimido[5,4-c]quinoline-2,4-diamine Derivative, and (ii) another anticancer agent are administered within 3 hours of each other. In another embodiment, i) a Pyrimido[5,4-c]quinoline-2,4-diamine Derivative, and (ii) another anticancer agent are administered 1 minute to 24 hours apart.

In one embodiment, an effective amount of a Pyrimido[5,4-c]quinoline-2,4-diamine Derivative and an effective amount of another anticancer agent are present in the same composition. In one embodiment, this composition is useful for oral administration. In another embodiment, this composition is useful for intravenous administration.

Cancers that can be treated or prevented by administering a Pyrimido[5,4-c]quinoline-2,4-diamine Derivative and another anticancer agent include, but are not limited to, the list of cancers set forth in Table 1.

In one embodiment the cancer is lung cancer, breast cancer, colorectal cancer, prostate cancer, a leukemia, a lymphoma, a skin cancer, a brain cancer, a cancer of the central nervous system, ovarian cancer, uterine cancer, stomach cancer, pancreatic cancer, esophageal cancer, kidney cancer, liver cancer, or a head and neck cancer.

The Pyrimido[5,4-c]quinoline-2,4-diamine Derivative and the other anticancer agent can act additively or synergistically. A synergistic combination of a Pyrimido[5,4-c]quinoline-2,4-diamine Derivative and another anticancer agent might allow the use of lower dosages of one or both of these agents and/or less frequent dosages of one or both of the Pyrimido[5,4-c]quinoline-2,4-diamine Derivatives and other anticancer agents and/or to administer the agents less frequently can reduce any toxicity associated with the administration of the agents to a subject without reducing the efficacy of the agents in the treatment of cancer. In addition, a synergistic effect might result in the improved efficacy of these agents in the treatment of cancer and/or the reduction of any adverse or unwanted side effects associated with the use of either agent alone.

In one embodiment, a Pyrimido[5,4-c]quinoline-2,4-diamine Derivative and another anticancer agent may act synergistically when administered in doses typically employed when such agents are used as monotherapy for the treatment of cancer. In another embodiment, a Pyrimido[5,4-c]quinoline-2,4-diamine Derivative and another anticancer agent may act synergistically when administered in doses that are less than doses typically employed when such agents are used as monotherapy for the treatment of cancer.

In one embodiment, the administration of an effective amount of a Pyrimido[5,4-c]quinoline-2,4-diamine Derivative and an effective amount of another anticancer agent inhibits the resistance of a cancer to the Pyrimido[5,4-c]quinoline-2,4-diamine Derivative and/or the other anticancer agent. In one embodiment, the cancer is a solid tumor.

In one embodiment, other anticancer agents useful in the methods and compositions of the present invention include, but are not limited to, a drug listed in Table 2 or a pharmaceutically acceptable salt thereof.

TABLE 2

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsulphonates: | Busulfan |
| | Treosulfan |
| Triazenes: | Dacarbazine |
| | Procarbazine |
| | Temozolomide |
| Platinum complexes: | Cisplatin |
| | Carboplatin |
| | Aroplatin |
| | Oxaliplatin |
| Plant Alkaloids | |
| Vinca alkaloids: | Vincristine |
| | Vinblastine |
| | Vindesine |
| | Vinorelbine |
| Taxanes: | Paclitaxel |
| | Docetaxel |
| DNA Topoisomerase Inhibitors | |
| Epipodophyllins: | Etoposide |
| | Teniposide |
| | Topotecan |
| | Irinotecan |
| | 9-aminocamptothecin |
| | Camptothecin |
| | Crisnatol |
| Mitomycins: | Mitomycin C |
| Anti-folates: | |
| DHFR inhibitors: | Methotrexate |
| | Trimetrexate |
| IMP dehydrogenase Inhibitors: | Mycophenolic acid |
| | Tiazofurin |
| | Ribavirin |
| | EICAR |
| Ribonuclotide reductase | Hydroxyurea |
| Inhibitors: | Deferoxamine |
| Pyrimidine analogs: | |
| Uracil analogs: | 5-Fluorouracil |
| | Fluoxuridine |
| | Doxifluridine |
| | Ralitrexed |
| Cytosine analogs: | Cytarabine |
| | Cytosine arabinoside |
| | Fludarabine |
| | Gemcitabine |
| | Capecitabine |
| Purine analogs: | Mercaptopurine |
| | Thioguanine |
| | O-6-benzylguanine |
| DNA Antimetabolites: | 3-HP |
| | 2′-deoxy-5-fluorouridine |
| | 5-HP |
| | alpha-TGDR |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | Pyrazoloimidazole |
| Hormonal therapies: | |
| Receptor antagonists: | |
| Anti-estrogen: | Tamoxifen |
| | Raloxifene |
| | Megestrol |
| LHRH agonists: | Goserelin |
| | Leuprolide acetate |
| Anti-androgens: | Flutamide |
| | Bicalutamide |
| Retinoids/Deltoids | Cis-retinoic acid |
| Vitamin A derivative: | All-trans retinoic acid (ATRA-IV) |
| Vitamin D3 analogs: | EB 1089 |
| | CB 1093 |
| | KH 1060 |
| Photodynamic therapies: | Vertoporfin (BPD-MA) |
| | Phthalocyanine |
| | Photosensitizer Pc4 |
| | Demethoxy-hypocrellin A |
| | (2BA-2-DMHA) |
| Cytokines: | Interferon-α |
| | Interferon-β |
| | Interferon-γ |
| | Tumor necrosis factor |
| | Interleukin-2 |
| Angiogenesis Inhibitors: | Angiostatin (plasminogen fragment) |
| | antiangiogenic antithrombin III |
| | Angiozyme |
| | ABT-627 |
| | Bay 12-9566 |
| | Benefin |
| | Bevacizumab |
| | BMS-275291 |
| | cartilage-derived inhibitor (CDI) |
| | CAI |
| | CD59 complement fragment |
| | CEP-7055 |
| | Col 3 |
| | Combretastatin A-4 |
| | Endostatin (collagen XVIII fragment) |
| | Fibronectin fragment |
| | Gro-beta |
| | Halofuginone |
| | Heparinases |
| | Heparin hexasaccharide fragment |
| | HMV833 |
| | Human chorionic gonadotropin (hCG) |
| | IM-862 |
| | Interleukins |
| | Kringle 5 (plasminogen fragment) |
| | Marimastat |
| | Metalloproteinase inhibitors |
| | 2-Methoxyestradiol |
| | MMI 270 (CGS 27023A) |
| | MoAb IMC-1C11 |
| | Neovastat |
| | NM-3 |
| | Panzem |
| | PI-88 |
| | Placental ribonuclease inhibitor |
| | Plasminogen activator inhibitor |
| | Platelet factor-4 (PF4) |
| | Prinomastat |
| | Prolactin 16 kD fragment |
| | Proliferin-related protein (PRP) |
| | PTK 787/ZK 222594 |
| | Retinoids |
| | Solimastat |
| | Squalamine |
| | SS 3304 |
| | SU 5416 |
| | SU6668 |
| | SU11248 |
| | Tetrahydrocortisol-S |
| | Tetrathiomolybdate |
| | Thalidomide |
| | Thrombospondin-1 (TSP-1) |
| | TNP-470 |
| | Transforming growth factor-beta |
| | (TGF-□) |
| | Vasculostatin |
| | Vasostatin (calreticulin fragment) |
| | ZD6126 |
| | ZD 6474 |
| | farnesyl transferase inhibitors (FTI) |
| | Bisphosphonates |
| Antimitotic agents: | Allocolchicine |
| | Halichondrin B |
| | Colchicine |
| | colchicine derivative |
| | dolastatin 10 |
| | Maytansine |
| | Rhizoxin |
| | Thiocolchicine |
| | trityl cysteine |
| Others: | |
| Isoprenylation inhibitors: | |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin |
| | Idarubicin |
| | Epirubicin |
| | Pirarubicin |
| | Zorubicin |
| | Mitoxantrone |
| MDR inhibitors: | Verapamil |
| Ca²⁺ ATPase inhibitors: | Thapsigargin |

Additional suitable other anticancer agents useful in the methods and compositions of the present invention include, but are not limited to abiraterone, acivicin, aclarubicin, acodazole, acronine, acylfulvene, adecypenol, adozelesin, aldesleukin, an ALL-TK antagonist, altretamine, ambamustine, ambomycin, ametantrone, amidox, amifostine, aminoglutethimide, aminolevulinic acid, amrubicin, amsacrine, anagrelide, anastrozole, andrographolide, an angiogenesis inhibitor, antarelix, anthramycin, an apoptosis gene modulator, apurinic acid, ara-CDP-DL-PTBA, arginine deaminase, L-asparaginase, asperlin, asulacrine, atamestane, atrimustine, axinastatin 1, axinastatin 2, axinastatin 3, azacitidine, azasetron, azatoxin, azetepa, azatyrosine, azotomycin, batimastat, benzodepa, bisantrene, bisnafide, bizelesin, brequinar, bropirimine, balanol, a BCR/ABL antagonist, beta-alethine, betaclamycin B, betulinic acid, bisaziridinylspermine, bisnafide, bistratene A, bizelesin, calcipotriol, calphostin C, calusterone, canarypox IL-2, carubicin, carboxyamidotriazole, CaRest M3, CARN 700, carzelesin, castanospermine, cecropin B, cetrorelix, chloroquinoxaline, cicaprost, cirolemycin, cladribine, clotrimazole, collismycin A, collismycin B, conagenin, crambescidin 816, crisnatol, cryptophycin 8, cryptophycin A derivatives, curacin A, cyclopentanthraquinones, cycloplatam, cypemycin, cytostatin, dacliximab, decitabine, dehydrodidemnin B, deslorelin, dexifosfamide, dexormaplatin, dexrazoxane, dexdiaziquone, didemnin B, didox, diethylnorspermine, dihydro-5-acytidine, dihydrotaxol, dioxamycin, diphenyl spiromustine, docosanol, dolasetron, droloxifene, dronabinol, duazomycin, duocarmycin SA, ecomustine, edatrexate, eflornithine, elsamitrucin, enloplatin, enpromate, epipropidine, erbulozole, esorubicin, estramustine, estramustine, an estrogen antagonist, etanidazole, etoprine, exemestane, fadrozole, fazarabine, fenretinide, finasteride, flavopiridol, flezelastine, fluasterone, fluorodaunorunicin, floxuridine, fluorocitabine, forfenimex, formestane, fostriecin, fotemustine, gadolinium texaphyrin, galocitabine, ganirelix, a gelatinase inhibitor, a glutathione inhibitor, hepsulfam, herbimycin A, heregulin, hexamethylene bisacetamide, hypericin, ibandronic acid, idoxifene, idramantone, ilmofosine, ilomastat, imatinib mesylate, imidazoacridones, imiquimod, an IGF-1 inhibitor, iobenguane, iodoipomeanol, iproplatin, irsogladine, isobengazole, isohomohalicondrin B, itasetron, jasplakinolide, leucovorin, levamisole, leuprorelin, liarozole, lissoclinamide 7, lobaplatin, lombricine, lometrexol, lonidamine, losoxantrone, lovastatin, loxoribine, lurtotecan, lutetium texaphyrin, lysofylline, mannostatin A, masoprocol, maspin, a matrix metalloproteinase inhibitor, mechlorethamine, megestrol acetate melphalan, metoclopramide, mifepristone, miltefosine, mirimostim, mitoguazone, mitolactol, mitonafide, mofarotene, molgramostim, mopidamol, a multiple drug resistance gene inhibitor, myriaporone, N-acetyldinaline, nafarelin, nagrestip, napavin, naphterpin, nartograstim, nedaplatin, nemorubicin, neridronic acid, nilutamide, nisamycin, a nitrogen mustard, a nitric oxide modulator, a nitrosourea, nitrullyn, nocodazole, octreotide, okicenone, onapristone, oracin, ormaplatin, osaterone, oxaunomycin, palauamine, palmitoylpamidronic acid, panaxytriol, panomifene, parabactin, pazelliptine, pegaspargase, peldesine, peliomycin, pentamustine, pentosan, pentostatin, pentrozole, peplomycin, perfosfamide, perflubron, perfosfamide, phenazinomycin, a phosphatase inhibitor, picibanil, pilocarpine, pipobroman, piposulfan, piritrexim, placetin A, placetin B, plicamycin, porfiromycin, plomestane, porfimer sodium, porfiromycin, prednimustine, prednisone, prostaglandin J2, microalgal, puromycin, pyrazoloacridine, pyrazofurin, a raf antagonist, raltitrexed, ramosetron, a ras farnesyl protein transferase inhibitor, a ras-GAP inhibitor, retelliptine demethylated, RII retinamide, riboprine, rogletimide, rohitukine, romurtide, roquinimex, rubiginone B1, ruboxyl, safingol, saintopin, SarCNU, sarcophytol A, sargramostim, semustine, a signal transduction modulator, simtrazene, sizofuran, sobuzoxane, solverol, sonermin, sparfosic acid, sparfosate, sparsomycin, spicamycin D, spiromustine, spiroplatin, splenopentin, spongistatin 1, a stem-cell division inhibitor, stipiamide, streptonigrin, a stromelysin inhibitor, sulfinosine, suradista, suramin, swainsonine, talisomycin, tallimustine, tauromustine, tazarotene, tecogalan, tegafur, tellurapyrylium, a telomerase inhibitor, teloxantrone, temoporfin, teroxirone, testolactone, tetrachlorodecaoxide, tetrazomine, thaliblastine, thiamiprine, thiocoraline, thrombopoietin, thymalfasin, thymotrinan, tirapazamine, titanocene, topsentin, toremifene, trestolone, tretinoin, triacetyluridine, triciribine, trimetrexate, triptorelin, tropisetron, tubulozole, turosteride, a tyrosine kinase inhibitor, ubenimex, uracil mustard, uredepa, vapreotide, variolin B, velaresol, veramine, verteporfin, vinxaltine, vinepidine, vinglycinate, vinleurosine, vinrosidine, vinzolidine, vitaxin, vorozole, zanoterone, zeniplatin, zilascorb, zinostatin, and zorubicin.

In various embodiments, the other anticancer agent is an alkylating agent, a platinum-containing agent, an anthracycline, a vinca alkaloid, a taxane, a topoisomerase inhibitor or an angiogenesis inhibitor.

In one embodiment, the other anticancer agent is administered orally.

In another embodiment, the other anticancer agent is administered intravenously.

### 5.4.4. Multi-Therapy for Cancer

The Pyrimido[5,4-c]quinoline-2,4-diamine Derivatives can be administered to a subject that has undergone or is currently undergoing one or more additional anticancer therapies including, but not limited to, surgery, radiation therapy, or immunotherapy, such as cancer vaccines.

In one embodiment, the invention provides methods for treating or preventing cancer comprising administering to a subject in need thereof (a) an amount of a Pyrimido[5,4-c]quinoline-2,4-diamine Derivative effective to treat or prevent cancer; and (b) another anticancer therapy including, but not limited to, surgery, radiation therapy, or immunotherapy, such as a cancer vaccine.

In one embodiment, the other anticancer therapy is radiation therapy.

In another embodiment, the other anticancer therapy is surgery.

In still another embodiment, the other anticancer therapy is immunotherapy.

In a specific embodiment, the present methods for treating or preventing cancer comprise administering a Pyrimido[5,4-c]quinoline-2,4-diamine Derivative and radiation therapy. The radiation therapy can be administered concurrently with, prior to, or subsequent to the Pyrimido[5,4-c]quinoline-2,4-diamine Derivative, in one embodiment, at least an hour, five hours, 12 hours, a day, a week, a month, or several months (e.g., up to three months), prior or subsequent to administration of the Pyrimido[5,4-c]quinoline-2,4-diamine Derivatives.

Where the other anticancer therapy is radiation therapy, any radiation therapy protocol can be used depending upon the type of cancer to be treated. For example, but not by way of limitation, X-ray radiation can be administered; in particular, high-energy megavoltage (radiation of greater that 1 MeV energy) can be used for deep tumors, and electron beam and orthovoltage X-ray radiation can be used for skin cancers. Gamma-ray emitting radioisotopes, such as radioactive isotopes of radium, cobalt and other elements, can also be administered.

Additionally, in one embodiment the invention provides methods of treatment of cancer using a Pyrimido[5,4-c]quinoline-2,4-diamine Derivative as an alternative to chemotherapy or radiation therapy where the chemotherapy or the radiation therapy results in negative side effects in the subject being treated. The subject being treated can, optionally, be treated with another anticancer therapy such as surgery, radiation therapy, or immunotherapy.

The Pyrimido[5,4-c]quinoline-2,4-diamine Derivatives can also be used in vitro or ex vivo, such as for the treatment of certain cancers, including, but not limited to leukemias and lymphomas, wherein such treatment involves autologous stem cell transplants. This can involve a process in which the subject's autologous hematopoietic stem cells are harvested and purged of all cancer cells, the subject's remaining bone-marrow cell population is then eradicated via the administration of a Pyrimido[5,4-c]quinoline-2,4-diamine Derivative and/or radiation, and the resultant stem cells are infused back into the subject. Supportive care can be subsequently provided while bone marrow function is restored and the subject recovers.

### 5.4.5. Modulation of Activity of PDK-1

Pyrimido[5,4-c]quinoline-2,4-diamine Derivatives are useful for modulating activity of PDK-1.

PDK-1 is believed to phosphorylate and activate several AGC family protein kinases, including isoforms of protein kinase B (PKB)/Akt, p70 ribosomal S6 kinase (S6K), serum- and glucocorticoid-induced protein kinase (SGK) and protein kinase C (PKC), which are involved in regulating physiological processes relevant to metabolism, growth, proliferation and survival. Modulation of PDK-1 activity can, therefore, effect treatment or prevention of diseases which result from an increase, decrease, or lack of activity or expression of PDK-1, PKB, S6K, SGK, PKC or other proteins involved in the AGC kinase signal transduction pathway.

### 5.5. Therapeutic/Prophylactic Administration

In one embodiment, the invention provides compositions useful for treating or preventing a proliferative disorder. The compositions are suitable for internal use and comprise an effective amount of a Pyrimido[5,4-c]quinoline-2,4-diamine Derivative and a physiologically acceptable carrier or vehicle.

The Pyrimido[5,4-c]quinoline-2,4-diamine Derivatives can be administered in amounts that are effective to treat or prevent a proliferative disorder in a subject.

Administration of the Pyrimido[5,4-c]quinoline-2,4-diamine Derivatives can be accomplished via any mode of administration for therapeutic agents. These modes include systemic or local administration such as oral, nasal, parenteral, transdermal, subcutaneous, vaginal, buccal, rectal or topical administration modes. In some instances, administration will result in the release of a Pyrimido[5,4-c]quinoline-2,4-diamine Derivative into the bloodstream.

In one embodiment, the Pyrimido[5,4-c]quinoline-2,4-diamine Derivatives are administered orally.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, preferably in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions include tablets and gelatin capsules comprising a Pyrimido[5,4-c]quinoline-2,4-diamine Derivative and a physiologically acceptable carrier or vehicle. Illustrative carriers or vehicles include a) a diluent, e.g., lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; and/or e) absorbent, colorant, flavorant and sweetener.

Liquid, particularly injectable, compositions can, for example, be prepared by dissolution, dispersion, etc. For example, the Pyrimido[5,4-c]quinoline-2,4-diamine Derivative is dissolved in or mixed with a pharmaceutically acceptable solvent such as, for example, water, saline, aqueous dextrose, glycerol, ethanol, and the like, to thereby form an injectable isotonic solution or suspension.

The Pyrimido[5,4-c]quinoline-2,4-diamine Derivatives can be also formulated as a suppository that can be prepared from fatty emulsions or suspensions; using polyalkylene glycols such as propylene glycol, as the carrier.

The Pyrimido[5,4-c]quinoline-2,4-diamine Derivatives can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

Pyrimido[5,4-c]quinoline-2,4-diamine Derivatives can also be delivered by the use of monoclonal antibodies as individual carriers to which the Pyrimido[5,4-c]quinoline-2,4-diamine Derivative molecules are coupled. The Pyrimido[5,4-c]quinoline-2,4-diamine Derivatives can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the Pyrimido[5,4-c]quinoline-2,4-diamine Derivatives can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Parental injectable administration can be used for subcutaneous, intramuscular or intravenous injections and infusions. Injectables can be prepared in conventional forms, either as liquid solutions or suspensions or solid forms suitable for dissolving in liquid prior to injection.

One embodiment, for parenteral administration employs the implantation of a slow-release or sustained-release system, according to U.S. Pat. No. 3,710,795, incorporated herein by reference.

The compositions can be sterilized or contain non-toxic amounts of adjuvants, such as preserving, stabilizing, wetting or emulsifying agents, solution promoters, salts for regulating the osmotic pressure pH buffering agents, and other substances, including, but not limited to, sodium acetate or triethanolamine oleate. In addition, they can also contain other therapeutically valuable substances.

Compositions can be prepared according to conventional mixing, granulating or coating methods, respectively, and the present pharmaceutical compositions can contain from about 0.1% to about 99%, preferably from about 1% to about 70% of the Pyrimido[5,4-c]quinoline-2,4-diamine Derivative by weight or volume.

The dosage regimen utilizing the Pyrimido[5,4-c]quinoline-2,4-diamine Derivative can be selected in accordance with a variety of factors including type, species, age, weight, sex and medical condition of the subject; the severity of the condition to be treated; the route of administration; the renal or hepatic function of the subject; and the particular Pyrimido[5,4-c]quinoline-2,4-diamine Derivative employed. A person skilled in the art can readily determine and prescribe the effective amount of the drug useful for treating or preventing a proliferative disorder.

Effective dosage amounts of the Pyrimido[5,4-c]quinoline-2,4-diamine Derivatives, when administered to a subject, range from about 0.05 to about 1000 mg of Pyrimido[5,4-c]quinoline-2,4-diamine Derivative per day. Compositions for in vivo or in vitro use can contain about 0.5, 1.0, 2.5, 5.0, 10.0, 15.0, 25.0, 50.0, 100.0, 250.0, 500.0 or 1000.0 mg of Pyrimido[5,4-c]quinoline-2,4-diamine Derivative. In one embodiment, the compositions are in the form of a tablet that can be scored. Effective plasma levels of the Pyrimido[5,4-c]quinoline-2,4-diamine Derivatives can range from about 0.002 mg to about 50 mg per kg of body weight per day. The amount of a Pyrimido[5,4-c]quinoline-2,4-diamine Derivative that is effective in the treatment or prevention of cancer can be determined by clinical techniques that are known to those of skill in the art. In addition, in vitro and in vivo assays can optionally be employed to help identify optimal dosage ranges. The precise dose to be employed can also depend on the route of administration, and the seriousness of the proliferative disorder being treated and can be decided according to the judgment of the practitioner and each subject's circumstances in view of, e.g., published clinical studies. Suitable effective dosage amounts, however, can range from about 10 micrograms to about 5 grams about every 4 h, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one Pyrimido[5,4-c]quinoline-2,4-diamine Derivative is administered, the effective dosage amounts correspond to the total amount administered.

The dosage regimen utilizing the Pyrimido[5,4-c]quinoline-2,4-diamine Derivative can be selected in accordance with a variety of factors including type, species, age, weight, sex and medical condition of the subject; the severity of the proliferative disorder to be treated; the route of administration; the renal or hepatic function of the subject; and the particular Pyrimido[5,4-c]quinoline-2,4-diamine Derivative employed. A person skilled in the art can readily determine and prescribe the effective amount of the drug required to prevent, counter or arrest the progress of the proliferative disorder.

Pyrimido[5,4-c]quinoline-2,4-diamine Derivatives can be administered in a single daily dose, or the total daily dosage can be administered in divided doses of two, three or four times daily. Furthermore, Pyrimido[5,4-c]quinoline-2,4-diamine Derivatives can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of Pyrimido[5,4-c]quinoline-2,4-diamine Derivative ranges from about 0.1% to about 15%, w/w or w/v.

In one embodiment, the compositions comprise an amount of each of a Pyrimido[5,4-c]quinoline-2,4-diamine Derivative and another anticancer agent which together are effective to treat or prevent cancer. In another embodiment, the amount of Pyrimido[5,4-c]quinoline-2,4-diamine Derivative and another anticancer agent is at least about 0.01% of the combined combination chemotherapy agents by weight of the composition. When intended for oral administration, this amount can be varied from about 0.1% to about 80% by weight of the composition. Some oral compositions can comprise from about 4% to about 50% of a Pyrimido[5,4-c]quinoline-2,4-diamine Derivative and another anticancer agent. Other compositions of the present invention are prepared so that a parenteral dosage unit contains from about 0.01% to about 2% by weight of the composition.

The Pyrimido[5,4-c]quinoline-2,4-diamine Derivatives can be assayed in vitro or in vivo for the desired therapeutic or prophylactic activity prior to use in humans. Animal model systems can be used to demonstrate safety and efficacy.

The present methods for treating or preventing cancer in a subject in need thereof can further comprise administering another prophylactic or therapeutic agent to the subject being administered a Pyrimido[5,4-c]quinoline-2,4-diamine Derivative. In one embodiment the other prophylactic or therapeutic agent is administered in an effective amount. The other prophylactic or therapeutic agent includes, but is not limited to, an anti-inflammatory agent, an anti-renal failure agent, an anti-diabetic agent, and anti-cardiovasculare disease agent, an antiemetic agent, a hematopoietic colony stimulating factor, an anxiolytic agent, and an analgesic agent.

In a further embodiment, the Pyrimido[5,4-c]quinoline-2,4-diamine Derivative can be administered prior to, concurrently with, or after an antiemetic agent, or on the same day, or within 1 hour, 2 hours, 12 hours, 24 hours, 48 hours or 72 hours of each other.

In another embodiment, the Pyrimido[5,4-c]quinoline-2,4-diamine Derivative can be administered prior to, concurrently with, or after a hematopoietic colony stimulating factor, or on the same day, or within 1 hour, 2 hours, 12 hours, 24 hours, 48 hours, 72 hours, 1 week, 2 weeks, 3 weeks or 4 weeks of each other.

In still embodiment, the Pyrimido[5,4-c]quinoline-2,4-diamine Derivative can be administered prior to, concurrently with, or after an opioid or non-opioid analgesic agent, or on the same day, or within 1 hour, 2 hours, 12 hours, 24 hours, 48 hours or 72 hours of each other.

In yet another embodiment, the Pyrimido[5,4-c]quinoline-2,4-diamine Derivative can be administered prior to, concurrently with, or after an anxiolytic agent, or on the same day, or within 1 hour, 2 hours, 12 hours, 24 hours, 48 hours or 72 hours of each other.

Effective amounts of the other therapeutic agents are well known to those skilled in the art. However, it is well within the skilled artisan's purview to determine the other therapeutic agent's optimal effective amount range. In one embodiment of the invention, where, another therapeutic agent is administered to a subject, the effective amount of the Pyrimido[5,4-c]quinoline-2,4-diamine Derivative is less than its effective amount would be where the other therapeutic agent is not administered. In this case, without being bound by theory, it is believed that the Pyrimido[5,4-c]quinoline-2,4-diamine Derivative and the other therapeutic agent act synergistically to treat or prevent cancer.

Antiemetic agents useful in the methods of the present invention include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinol, thiethylperazine, thioproperazine, and tropisetron.

Hematopoietic colony stimulating factors useful in the methods of the present invention include, but are not limited to, filgrastim, sargramostim, molgramostim and epoietin alfa.

Opioid analgesic agents useful in the methods of the present invention include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene.

Non-opioid analgesic agents useful in the methods of the present invention include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

Anxiolytic agents useful in the methods of the present invention include, but are not limited to, buspirone, and benzodiazepines such as diazepam, lorazepam, oxazapam, chlorazepate, clonazepam, chlordiazepoxide and alprazolam.

### 5.6. Kits

The invention encompasses kits that can simplify the administration of a Pyrimido[5,4-c]quinoline-2,4-diamine Derivative to a subject.

A typical kit of the invention comprises a unit dosage form of a Pyrimido[5,4-c]quinoline-2,4-diamine Derivative. In one embodiment the unit dosage form is a container, which can be sterile, containing an effective amount of a Pyrimido[5,4-c]quinoline-2,4-diamine Derivative and a physiologically acceptable carrier or vehicle. The kit can further comprise a label or printed instructions instructing the use of the Pyrimido[5,4-c]quinoline-2,4-diamine Derivative to treat or prevent cancer. The kit can also further comprise a unit dosage form of another prophylactic or therapeutic agent, for example, a container containing an effective amount of another prophylactic or therapeutic agent or another anticancer agent. In one embodiment the kit comprises a container containing an effective amount of a Pyrimido[5,4-c]quinoline-2,4-diamine Derivative and an effective amount of another prophylactic or therapeutic agent. Examples of other prophylactic or therapeutic agents and other anticancer agents include, but are not limited to, those listed above.

Kits of the invention can further comprise a device that is useful for administering the unit dosage forms. Examples of such a device include, but are not limited to, a syringe, a drip bag, a patch, an inhaler, and an enema bag. The invention is further described in the following examples, which do not limit the scope of the invention described in the claims.

The following examples illustrate the synthesis of illustrative Pyrimido[5,4-c]quinoline-2,4-diamine Derivatives and demonstrate their usefulness for treating or preventing a proliferative disorder.

### 5. EXAMPLES

### Example 1

### Synthesis of Compound Ia-6

Step A: Preparation of 3-Methoxy-4-(2-methoxy-ethoxy)-Benzoic Acid Methyl Ester

A mixture of 4-hydroxy-3-methoxy-benzoic acid methyl ester (Aldrich Chemical Co.) (100 g, 549 mmol), 1-bromo-2-methoxy-ethane (76.3, 549 mmol), aliquat-336 (Aldrich Chemical Co.), (2 g), and potassium carbonate (113.8, 823 mmol) is stirred and refluxed in 1 L of acetone for 18 hours. The mixture is filtered and the solvent is removed. The residue is re-dissolved in ethyl acetate and the solution is washed with a dilute base. The solution is dried (using MgSO₄) and filtered through a pad of hydrous magnesium silicate (Brand-Nu Laboratories). Solvent is removed to provide the title compound (128 g).

Step B: Preparation of 5-Methoxy-4-(2-methoxy-ethoxy)-2-nitro-benzoic Acid Methyl Ester

3-Methoxy-4-(2-methoxy-ethoxy)-benzoic acid methyl ester (128 g, 532.8 mmol) is dissolved in 364 ml of acetic acid to which 145 ml of concentrated nitric acid is added dropwise over 35 minutes. The mixture is stirred for 20 hours and then poured into water. The solid product is collected and washed with water and hexanes to provide the title compound after drying (149 g).

Step C: Preparation of 2-Amino-5-methoxy-4-(2-methoxy-ethoxy)-benzoic Acid Methyl Ester

A mixture of 5-methoxy-4-(2-methoxy-ethoxy)-2-nitro-benzoic acid methyl ester (149 g, 522.2 mmol) powdered iron (116.7 g, 2 mmol), and ammonium chloride (139.7 g, 2.61 mmol) in 500 ml of water and 1500 ml of methanol is stirred at reflux for 2 hours. The hot mixture is filtered and the organic solvents are removed. A solution of sodium bicarbonate is added to the residue and the mixture is extracted with ethyl acetate. The extract is dried (using MgSO₄) and filtered through a pad of hydrous magnesium silicate. The solution is concentrated to about 500 ml and then diluted with about 500 ml of hexanes. The mixture is cooled and the product solid is collected to provide 86.8 g of the title compound.

Step D: Preparation of 2-(dimethylamino-methyleneamino)-5-methoxy-4-(2-methoxy-ethoxy)-benzoic Acid Methyl Ester

A mixture of 2-amino-5-methoxy-4-(2-methoxy-ethoxy)-benzoic acid methyl ester (86.8 g, 340 mmol) and DMF-acetal (60.8, 67.8 mmol) is refluxed for 2 hours. The excess reagent is removed by distillation to provide 98.2 g of the title compound.

Step E: Preparation of 4-Hydroxy-6-methoxy-7-(2-methoxy-ethoxy)-quinoline-3-carbonitrile

To 135.3 ml (338.3 mmol) of 2.5 M n-butyl lithium in hexanes at −78° C., a solution of acetonitrile in 450 ml of THF is added dropwise over 45 min. After another 45 min, a solution of 2-(dimethylamino-methyleneamino)-5-methoxy-4-(2-methoxy-ethoxy)-benzoic acid methyl ester (50 g, 161.1 mmol) in 450 ml of THF is added over 1 hour. The mixture is warmed slowly to −55° C. and 46 ml of acetic acid is added. The mixture is warmed to room temperature and the solvent is removed. The residue is diluted with water and the resulting solid is collected, washed with water and hexanes and dried in vacuum to provide 44 g of the title compound.

Step F: Preparation of 4-Chloro-6-methoxy-7-(2-methoxy-ethoxy)-quinoline-3-carbonitrile

A mixture of hydroxy-6-methoxy-7-(2-methoxy-ethoxy)-quinoline-3-carbonitrile (40.2 g, 146.6 mmol), oxalyl chloride (83.7 g, 4.5 mmol), and DMF (1 g, 14.5 mmol) in 400 ml of methylene chloride is refluxed for 3.5 hrs. The mixture is filtered and the solvent is removed. The residue is re-dissolved in methylene chloride and the solution is washed with ice-cold sodium bicarbonate solution. The solution is dried (using MgSO₄) and filtered through a pad of hydrous magnesium silicate. Solvent is removed. The residue is dissolved in warm ethyl acetate and then diluted with hexanes and cooled. Product solid is collected to provide 29.4 g of the title compound.

Step G: Preparation of 3-Hydroxy-pyrrolidine-1-carboxamidine Sulfate Salt

A mixture of 3-hydroxy pyrrolidine (2.65 g, 30.4 mmol) and 2-methyl-2-thiopseudourea sulfate (4.23 g, 15.21 mmol) in 21 ml of water is stirred at 90° C. for 1 hour. About ⅔ of the water is removed under vacuum and the residue is diluted with ethanol. The resulting solid is collected and dried in vacuum to provide 4.4 grams of the title compound. Other guanidine sulfate salts used to prepare the compounds of this invention are prepared in a similar manner.

Step H: Preparation of 1-[4-Amino-9-methoxy-8-(2-methoxyethoxy)pyrimido[5,4-c]quinolin-2-yl]pyrrolidin-3-ol (Compound Ia-6)

A mixture of 3-hydroxy-pyrrolidine-1-carboxamidine sulfate salt (658 mg, 1.84 mmol) and 4-chloro-6-methoxy-7-(2-methoxy-ethoxy)-quinoline-3-carbonitrile (720 mg, 2.46 mmol) in 8 ml of t-butanol is refluxed with stirring for 20 hours. The mixture is cooled and diluted with water. The solid is collected and recrystallized from methanol to provide 310 mg of the title compound.

### Example 2

### Synthesis of other Illustrative Pyrimido[5,4-c]quinoline-2,4-diamine Derivatives

Compounds described in Table 3 are prepared as described in Step H of Example 1, by reaction of an appropriate guanidine acid salt, which is prepared as described in Step G of Example 1 or is purchased, and an appropriate 4-chloro-quinoline-3-carbonitrile, which prepared, for example, using methodology of Steps A-F. Compounds are then characterized using mass spectrometry (MS).

TABLE 3

| | | | MS: |
|---|---|---|---|
| | | | m/e |
| | | MS: m/e | (M + |
| Compound | Compound name | (M + H)⁺¹ | 2H⁾⁺² |
| Ia-1 | 8,9-dimethoxypyrimido[5,4-c]quinoline- | 272.1 | |
| | 2,4-diamine | | |
| Ia-2 | 8,9-diethoxypyrimido[5,4-c]quinoline- | 300.1 | |
| | 2,4-diamine | | |
| Ia-3 | 8-fluoro-9-methoxypyrimido[5,4- | 260.1 | |
| | c]quinoline-2,4-diamine | | |
| Ia-4 | 8,9-diethoxy-2-morpholin-4- | 370.1 | |
| | ylpyrimido[5,4-c]quinolin-4-amine | | |
| Ia-5 | 9-methoxy-8-(2-methoxyethoxy)-2- | 386.2 | |
| | morpholin-4-ylpyrimido[5,4-c]quinolin- | | |
| | 4-amine | | |
| Ia-7 | 9-methoxy-8-(2-methoxyethoxy)-2-(4- | 399.3 | 200.1 |
| | methylpiperazin-1-yl)pyrimido[5,4- | | |
| | c]quinolin-4-amine | | |
| Ia-8 | 9-methoxy-8-(2-methoxyethoxy)- | 344.2 | |
| | N2,N2-dimethylpyrimido[5,4- | | |
| | c]quinoline-2,4-diamine | | |
| Ia-9 | N2-ethyl-9-methoxy-8-(2- | 344.2 | |
| | methoxyethoxy)pyrimido[5,4- | | |
| | c]quinoline-2,4-diamine | | |
| Ia-10 | 2-(1,4-dioxa-8-azaspiro[4.5]dec-8-yl)-9- | 442.3 | |
| | methoxy-8-(2- | | |
| | methoxyethoxy)pyrimido[5,4- | | |
| | c]quinolin-4-amine | | |
| Ia-11 | N2-(4-chlorophenyl)-9-methoxy-8-(2- | 426.2 | |
| | methoxyethoxy)pyrimido[5,4- | | |
| | c]quinoline-2,4-diamine | | |
| Ia-12 | {1-[4-amino-9-methoxy-8-(2- | 414.3 | |
| | methoxyethoxy)pyrimido[5,4- | | |
| | c]quinolin-2-yl]piperidin-3-yl}methanol | | |
| Ia-13 | 1-[4-amino-9-methoxy-8-(2- | 398.2 | |
| | methoxyethoxy)pyrimido[5,4- | | |
| | c]quinolin-2-yl]piperidin-4-one | | |
| Ia-14 | 9-methoxy-8-(2-methoxyethoxy)-2- | 370.2 | |
| | pyrrolidin-1-ylpyrimido[5,4-c]quinolin- | | |
| | 4-amine | | |
| Ia-15 | 9-methoxy-8-(2-methoxyethoxy)-2- | 414.2 | 207.6 |
| | [(2R)-2-(methoxymethyl)pyrrolidin-1- | | |
| | yl]pyrimido[5,4-c]quinolin-4-amine | | |
| Ia-16 | 9-methoxy-8-(2-methoxyethoxy)-2- | 414.2 | 207.6 |
| | [(2S)-2-(methoxymethyl)pyrrolidin-1- | | |
| | yl]pyrimido[5,4-c]quinolin-4-amine | | |
| Ia-17 | N2-cyclohexyl-9-methoxy-8-(2- | 398.2 | |
| | methoxyethoxy)pyrimido[5,4- | | |
| | c]quinoline-2,4-diamine | | |
| Ia-18 | 9-methoxy-8-(2-methoxyethoxy)-2-(4- | 461.2 | 231.1 |
| | phenylpiperazin-1-yl)pyrimido[5,4- | | |
| | c]quinolin-4-amine | | |
| Ia-19 | 9-methoxy-8-(2-methoxyethoxy)-2-(4- | 460.2 | |
| | phenylpiperidin-1-yl)pyrimido[5,4- | | |
| | c]quinolin-4-amine | | |
| Ia-20 | 2-(2,3-dihydro-1,4-benzoxazepin-4(5H)- | 448.2 | |
| | yl)-9-methoxy-8-(2- | | |
| | methoxyethoxy)pyrimido[5,4- | | |
| | c]quinolin-4-amine | | |
| Ia-21 | N2-(3-chlorophenyl)-9-methoxy-8-(2- | 426.1 | |
| | methoxyethoxy)pyrimido[5,4- | | |
| | c]quinoline-2,4-diamine | | |
| Ia-22 | 2-[3-(1,3-dihydro-2H-isoindol-2- | 487.2 | 244.1 |
| | yl)pyrrolidin-1-yl]-9-methoxy-8-(2- | | |
| | methoxyethoxy)pyrimido[5,4- | | |
| | c]quinolin-4-amine | | |
| Ia-23 | N2-benzyl-9-methoxy-8-(2- | 406.2 | |
| | methoxyethoxy)pyrimido[5,4- | | |
| | c]quinoline-2,4-diamine | | |

### Example 3

### PDK-1 Kinase Inhibition by Pyrimido[5,4-c]quinoline-2,4-diamine Derivatives

The ability of an illustrative Pyrimido[5,4-c]quinoline-2,4-diamine Derivative to inhibit PDK-1 in vitro can be determined using an ELISA assay as described in Kobayashi et al., Biochem. J. 339:319-328 (1999) and Park et al., EMBO J. 18:3024-3033 (1999).

In an in vitro assay, His₆-tagged SGK1 with S422D mutation (formerly Upstate Biotech, now Millipore) on Thr256 is allowed to be phosphorylated by PDK-1 in the presence of a Pyrimido[5,4-c]quinoline-2,4-diamine Derivative. After 20 minutes, the reaction is quenched via the addition of urea. The His₆-tagged SGK1 is then bound to 96 well Ni-NTA HisSorb plates (Quiagen, Inc.) and phosphorylated SGK1 is quantitatively detected using rabbit derived anti-phospho SGK antibody (Cell Signaling Technology) as the primary antibody and horseradish peroxidase (HRP)— coupled anti-rabbit antibody (formerly Zymed, now Invitrogen) as the secondary antibody. HRP is then detected using a chemiluminescent substrate (Pierce Chemical Co.).

IC₅₀ values of illustrative Pyrimido[5,4-c]quinoline-2,4-diamine Derivatives, shown in Table 4, are calculated using LSW Data Analysis package add-in for Excel (Microsoft).

TABLE 4

| | Average |
|---|---|
| Compound | IC₅₀ [nm] |
| Ia-1 | 12,202 |
| Ia-2 | 17,965 |
| Ia-4 | 55,661 |
| Ia-5 | >200,000 |
| Ia-6 | 36,284 |
| Ia-7 | >200,000 |
| Ia-8 | 9,335 |
| Ia-9 | >200,000 |
| Ia-10 | 50,478 |
| Ia-11 | >200,000 |
| Ia-12 | 30,366 |
| Ia-13 | >200,000 |
| Ia-14 | 6,550 |
| Ia-15 | 2,145 |
| Ia-16 | 39,557 |
| Ia-17 | 5,431 |
| Ia-18 | 110,522 |
| Ia-20 | 83,122 |
| Ia-21 | 21,702 |

The results in Table 4 demonstrate that illustrative Pyrimido[5,4-c]quinoline-2,4-diamine Derivatives are effective at inhibiting PDK-1. Accordingly, a Pyrimido[5,4-c]quinoline-2,4-diamine Derivative is useful for treating or preventing a proliferative disorder, such as cancer, as well as for modulating PDK-1 activity.

The present invention is not to be limited in scope by the specific embodiments disclosed in the examples, which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosures of which have been incorporated herein in their entirety.

What is claimed is:

1. A compound having the formula: 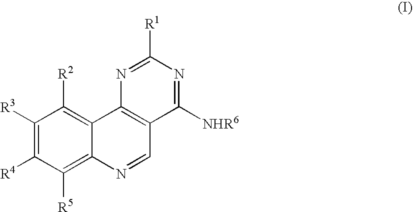
or a pharmaceutically acceptable salt thereof,
wherein
R¹ is —H, —N(R¹²)₂, —R¹³, —NR(C(R¹²)₂)ₙ—OR¹², —NR¹²—(C(R¹²)₂)ₙ—H, —NR¹²—(C(R¹²)₂)ₙ—R¹³, —NR¹²—(C(R¹²)₂)ₙ—N(R¹²)₂, —NR¹²—(C(R¹²)₂)ₙ—NR¹²—(C(R¹²)₂)ₙ—N(R¹²)₂, NR¹²—(C(R¹²)₂)ₙ—NR¹²—(C(R¹²)₂)ₙ—OR¹²;
n=1-4 at each occurrence, with the proviso that when the carbon chain of length n is between two heteroatoms, n=2-4;
R² and R⁵ are each independently —H, —OH-halo, —CN, —N₃, —NH₂, —NH—C₁-C₆ alkyl, —N(C₁-C₆ alkyl)₂, —C₁-C₆ alkyl, —O—C₁-C₆ alkyl, or —S—C₁-C₆ alkyl, wherein each C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R³ and R⁴ are each independently —H, —OH, -halo, —NH₂, —CN, —NO₂, —COOH, —C₁-C₆ alkyl, —C₂-C₆ alkenyl, —C₂-C₆ alkynyl, —O—C₁-C₆ alkyl, —O—C₂-C₆ alkenyl, —O—C₂-C₆ alkynyl, —C(O)O—C₁-C₆ alkyl, —C(O)NH—C₁-C₆ alkyl, —C(O)N(C₁-C₆ alkyl)₂, —C₁-C₆-alkylene-O—C₁-C₆ alkyl, —O—C₁-C₆-alkylene-O—C₁-C₆ alkyl, —C₁-C₆-alkylene-C(O)O—C₁-C₆ alkyl, —C₁-C₆-alkylene-C(O)NH—C₁-C₆ alkyl, —OC(O)—C₁-C₆ alkyl, —OC(O)—C₂-C₆ alkenyl, —OC(O)—C₂-C₆ alkynyl, —C₁-C₆-alkylene-OC(O)—C₁-C₆ alkyl, —C₁-C₆ alkylene-OC(O)—C₂-C₆ alkenyl, —C₁-C₆ alkylene-OC(O)—C₂-C₆ alkynyl, —S—C₁-C₆ alkyl, —S(O)—C₁-C₆ alkyl, —S(O)₂—C₁-C₆ alkyl, —S(O)₂NH—C₁-C₆ alkyl, —S(O)₂NH—C₂-C₆ alkenyl, —S(O)₂NH—C₂-C₆ alkynyl, —C(O)-benzyl, —C(O)O—C₁-C₆ alkyl, —C(O)—C₁-C₆ alkyl, -phenyl, —O-phenyl, —S-phenyl, -benzyl, —O-benzyl, —CHO, —NHC(O)H, —NHOH, —NH—O—C₁-C₆ alkyl, —NH—C₁-C₆ alkyl, —N(C₁-C₆ alkyl)₂, —NHC(O)—C₁-C₆ alkyl, —N(C₁-C₆ alkyl)(C₂-C₆ alkenyl), —N(C₂-C₆ alkenyl)₂, —NH-phenyl, —NH-benzyl, 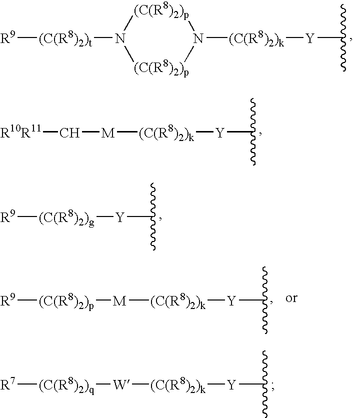
wherein
a=0-1;
g=1-6;
k=0-4;
p=2-4 at each occurrence;
q=0-4;
t=2-4;
M is a bond, —NR⁸—, —O—, —N((C(R⁸)₂)ₚN(R⁸)₂)—, —N((C(R⁸)₂)ₚOR⁸)—, —CC—, —CH═CH—, or phenylene;
W′ is a bond, —N(R⁸)—, —O—, —CC—, —CH═CH—, or phenylene;
Y is —(CH₂)ₐ—, —O—, —S—, —C(O)N(R⁸)—, —C(S)N(R⁸), or —NR⁸—;
when M is phenylene then p=0-4 and r=0-4;
when M is —O— and R⁹ is —OR⁸ then p=1-4;
when M is —CC— or —CH═CH— and Y is —S—, —O— or —NR⁸— then k=1-4;
when M is —CC— or —CH═CH— then p=1-4;
when W′ is —CC— or —CH═CH— and R⁷ is bonded through a heteroatom then q=1-4;
when W′ is a bond and q=0 and R⁷ is bonded through a N atom and Y is —S—, —O— or —NR⁸-then k=2-4;
when W′ is a bond and k=0 and R⁷ is bonded through a N atom and Y is —S—, —O— or —NR⁸-then q=2-4;
when W′ is not a bond, —CC—, —CH═CH—, or phenylene and R⁷ is bonded through a N atom then q=2-4;
when Y is —NR⁸— and R⁹ is —N(R⁸)₂, —N(R⁸)₃⁺ or —NR⁸(OR⁸) then g=2-6;
when Y is —N(R⁸)— and M is —N(R⁸)—, —O—, —N((C(R⁸)₂)ₚN(R⁸)₂— or —N((C(R⁸)₂)ₚ—OR⁸)— then k=2-4;
when Y is —N(R⁸)— and W′ is —N(R⁸)— or —O— then k=2-4;
when Y is —O— and either M or W′ is —O— then k=1-4;
wherein each —C₁-C₆ alkyl, —C₁-C₆ alkylene, —C₂-C₆ alkenyl, —C₂-C₆ alkynyl, -phenyl, -benzyl, or phenylene is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R⁶ is —H, —OH, or —C₁-C₆ alkyl, wherein each —C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R⁷ is -phenyl, a 5- or 6-membered aromatic monocyclic heterocycle which is unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl; or
R⁷ is a 3- to 7-membered non-aromatic monocyclic heterocycle which is unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl, benzoyl, -oxo or -thio,
wherein each R⁷ is independently unsubstituted or mono or di-substituted on a C atom with —R⁸, —N(R⁸)₂, —OR⁸, —(C(R⁸)₂)ᵣOR⁸, or —(C(R⁸)₂)ᵣN(R⁸)₂, or a 3- to 7-membered monocyclic heterocycle,
wherein r=1-6; or
each R⁷ is independently unsubstituted or mono or di-substituted on a saturated C atom with —O(C(R⁸)₂)ᵣO—,
wherein r=1-6; or
each R⁷ is independently unsubstituted or mono-substituted on a N atom with —R⁸;
R⁸ is each independently —H, —C₁-C₆ alkyl, —C₂-C₆ alkenyl, —C₂-C₆ alkynyl, —C₃-C₈ monocyclic cycloalkyl, —C(O)—C₁-C₆ alkyl, —C(O)O—C₁-C₆ alkyl, or -phenyl, wherein each —C₁-C₆ alkyl —C₂-C₆ alkenyl, —C₂-C₆ alkynyl, —C₃-C₈ monocyclic cycloalkyl or -phenyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R⁹ is -halo, —N(R⁸)₂, —OR⁸, —N(R⁸)₃⁺, or —NR⁸(OR⁸);
R¹⁰ and R¹¹ are each independently —(C(R⁸)₂)ₛN(R⁸)₂ or —(C(R⁸)₂)ₛOR⁸,
wherein s=1-4;
R¹² is each independently —H, —C₁-C₆ alkyl, —C₂-C₆ alkenyl, —C₂-C₆ alkynyl, —C₃-C₈ monocyclic cycloalkyl, —C₃-C₈ monocyclic cycloalkenyl, -phenyl, -benzyl, a 5- or 6-membered aromatic monocyclic heterocycle, or a 3- to 7-membered non-aromatic monocyclic heterocycle which is unsubstituted or substituted with one or more of —OH, -halo, —NH₂, —CN, —C₁-C₆ alkyl, -oxo or -thio, wherein each —C₁-C₆ alkyl, —C₂-C₆ alkenyl, —C₂-C₆ alkynyl, —C₃-C₈ monocyclic cycloalkyl, -phenyl, or benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹³ is a 5- or 6-membered aromatic monocyclic heterocycle which is unsubstituted or substituted with one or more of —OH, -halo, —NH₂, —CN or —C₁-C₆ alkyl, a 3- to 7-membered non-aromatic monocyclic heterocycle which is unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl, benzoyl, -oxo or -thio, or a 8- to 12-membered bicyclic heterocycle which is unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl, benzoyl, -oxo or -thio,
wherein each R¹³ is independently unsubstituted or mono- or di-substituted with 4-(2,3-dihydro-indol-1-yl), 4-(1,3-dihydro-isoindol-2-yl), —NH-benzoyl, a 5- or 6-membered aromatic monocyclic heterocycle, a 3- to 7-membered non-aromatic monocyclic heterocycle, 8- to 12-membered bicyclic heterocycle, —O-5- or 6-membered aromatic monocyclic heterocycle, —O-3- to 7-membered non-aromatic monocyclic heterocycle, —O-8- to 12-membered bicyclic heterocycle, or R¹⁴,
wherein each -benzoyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹⁴ is —H, -halo, —OH, —CN, —NH₂, —NO₂, —N₃, —COOH, —C₁-C₆ alkyl, —C₂-C₆ alkenyl, —C₂-C₆ alkynyl, —N₃, —NHC(O)—C₁-C₆ alkyl, —C₁-C₆ alkyl —O—C₁-C₆ alkyl, —C₁-C₆ alkylene-C(O)O—C₁-C₆ alkyl, —O—C₁-C₆ alkyl, —S—C₁-C₆ alkyl, —C(O)O—C₁-C₆ alkyl, —C(O)—C₁-C₆ alkyl, -phenyl, —O-phenyl, —S-phenyl, -benzyl, -benzoyl, —C₁-C₆ alkylene-C(O)O-phenyl, —NH—C₁-C₆ alkyl, —N(C₁-C₆ alkyl)₂, —NH-phenyl, —NH-benzyl, —NH—C(O)—C₁-C₆ alkyl, —NH—C(O)—C₂-C₆ alkenyl, —NH—C(O)—C₂-C₆ alkynyl, —C₁-C₆ alkylene-COOH, —C₁-C₆ alkylene-CHO, —C₁-C₆ alkylene-NH—C₁-C₆ alkyl, —C₁-C₆ alkylene-N(C₁-C₆ alkyl)₂, —C(O)NH—C₁-C₆ alkyl, —C(O)N(C₁-C₆ alkyl)₂, —O—C₁-C₆ alkylene-NH—C₁-C₆ alkyl, —O—C₁-C₆ alkylene-N(C₁-C₆ alkyl)₂, —OS(O)₂—C₁-C₆ alkyl, or —SH, wherein each —C₁-C₆ alkyl, —C₁-C₆ alkylene, —C₂-C₆ alkenyl, —C₂-C₆ alkynyl, -phenyl, -benzyl, or benzoyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl.

2. The compound or a pharmaceutically acceptable salt of a compound of claim 1, wherein R², R⁵ and/or R⁶ is —H.

3. The compound or a pharmaceutically acceptable salt of a compound of claim 1, wherein one or both of R³ and R⁴ is —O—C₁-C₆ alkyl.

4. The compound or a pharmaceutically acceptable salt of a compound of claim 1, wherein R⁴ is —O—C₁-C₆ alkylene-O—C₁-C₆ alkyl.

5. The compound or a pharmaceutically acceptable salt of a compound of claim 1, wherein R¹ is —NH₂.

6. The compound or a pharmaceutically acceptable salt of a compound of claim 1, wherein R¹ is —N(C₁-C₆ alkyl)₂.

7. The compound or a pharmaceutically acceptable salt of a compound of claim 1, wherein R¹ is —N(R¹²)₂.

8. The compound or a pharmaceutically acceptable salt of a compound of claim 7, wherein one or more R¹² is —H.

9. The compound or a pharmaceutically acceptable salt of a compound of claim 7, wherein one or more R¹² is —C₃-C₈ monocyclic cycloalkyl.

10. The compound or a pharmaceutically acceptable salt of a compound of claim 7, wherein one or more R¹² is -benzyl.

11. The compound or a pharmaceutically acceptable salt of a compound of claim 1, wherein R¹ is morpholino.

12. The compound of claim 1, wherein the compound is:
8,9-dimethoxypyrimido[5,4-c]quinoline-2,4-diamine;
8,9-diethoxypyrimido[5,4-c]quinoline-2,4-diamine;
8-fluoro-9-methoxypyrimido[5,4-c]quinoline-2,4-diamine;
8,9-diethoxy-2-morpholin-4-ylpyrimido[5,4-c]quinolin-4-amine;
9-methoxy-8-(2-methoxyethoxy)-2-morpholin-4-ylpyrimido[5,4-c]quinolin-4-amine;
1-[4-amino-9-methoxy-8-(2-methoxyethoxy)pyrimido[5,4-c]quinolin-2-yl]pyrrolidin-3-ol;
9-methoxy-8-(2-methoxyethoxy)-2-(4-methylpiperazin-1-yl)pyrimido[5,4-c]quinolin-4-amine;
9-methoxy-8-(2-methoxyethoxy)-N²,N²-dimethylpyrimido[5,4-c]quinoline-2,4-diamine;
N²-ethyl-9-methoxy-8-(2-methoxyethoxy)pyrimido[5,4-c]quinoline-2,4-diamine;
2-(1,4-dioxa-8-azaspiro[4.5]dec-8-yl)-9-methoxy-8-(2-methoxyethoxy)pyrimido[5,4-c]quinolin-4-amine;
N²-(4-chlorophenyl)-9-methoxy-8-(2-methoxyethoxy)pyrimido[5,4-c]quinoline-2,4-diamine;
{1-[4-amino-9-methoxy-8-(2-methoxyethoxy)pyrimido[5,4-c]quinolin-2-yl]piperidin-3-yl}methanol;
1-[4-amino-9-methoxy-8-(2-methoxyethoxy)pyrimido[5,4-c]quinolin-2-yl]piperidin-4-one;
9-methoxy-8-(2-methoxyethoxy)-2-pyrrolidin-1-ylpyrimido[5,4-c]quinolin-4-amine;
9-methoxy-8-(2-methoxyethoxy)-2-[(2R)-2-(methoxymethyl)pyrrolidin-1-yl]pyrimido[5,4-c]quinolin-4-amine;
9-methoxy-8-(2-methoxyethoxy)-2-[(2S)-2-(methoxymethyl)pyrrolidin-1-yl]pyrimido[5,4-c]quinolin-4-amine;
N²-cyclohexyl-9-methoxy-8-(2-methoxyethoxy)pyrimido[5,4-c]quinoline-2,4-diamine;
9-methoxy-8-(2-methoxyethoxy)-2-(4-phenylpiperazin-1-yl)pyrimido[5,4-c]quinolin-4-amine;
9-methoxy-8-(2-methoxyethoxy)-2-(4-phenylpiperidin-1-yl)pyrimido[5,4-c]quinolin-4-amine;
2-(2,3-dihydro-1,4-benzoxazepin-4(5H)-yl)-9-methoxy-8-(2-methoxyethoxy)pyrimido[5,4-c]quinolin-4-amine;
N²-(3-chlorophenyl)-9-methoxy-8-(2-methoxyethoxy)pyrimido[5,4-c]quinoline-2,4-diamine;
2-[3-(1,3-dihydro-2H-isoindol-2-yl)pyrrolidin-1-yl]-9-methoxy-8-(2-methoxyethoxy)pyrimido[5,4-c]quinolin-4-amine; or
N²-benzyl-9-methoxy-8-(2-methoxyethoxy)pyrimido[5,4-c]quinoline-2,4-diamine,
or a pharmaceutically acceptable salt thereof.

13. A composition comprising an effective amount of a compound or a pharmaceutically acceptable salt of a compound of claim 1 and a physiologically acceptable vehicle.

14. A solid dosage form comprising the composition of claim 13.

15. A method for treating a proliferative disorder, the method comprising administering to a subject in need thereof an effective amount of the compound or a pharmaceutically acceptable salt of the compound of claim 1.

16. The method of claim 15, wherein the proliferative disorder is cancer.

17. The method of claim 16, wherein the cancer is lung cancer, breast cancer, colorectal cancer, prostate cancer, a leukemia, a lymphoma, a skin cancer, a brain cancer, a cancer of the central nervous system, ovarian cancer, uterine cancer, stomach cancer, pancreatic cancer, esophageal cancer, kidney cancer, liver cancer, or a head and neck cancer.

18. The method of claim 16 further comprising the administration of another anticancer agent.

19. The method of claim 15, wherein the subject is a human.

20. A method for modulating activity of PDK-1, the method comprising administering to a subject in need thereof an effective amount of the compound or a pharmaceutically acceptable salt of the compound of claim 1, wherein it is known that PDK-1 activity is related to a disease or disorder.

21. The method of claim 20, wherein the disease or disorder is a proliferative disorder.

22. The method of claim 21, wherein the proliferative disorder is cancer.

23. A process for the preparation of a compound of Formula (I) 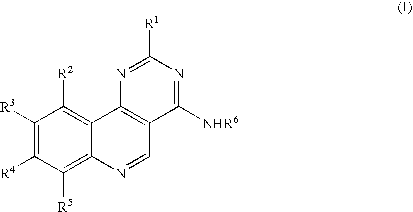
wherein
R¹ is —H, —N(R¹²)₂, —R¹³, —NR¹²—(C(R¹²)₂)ₙ—OR¹², —NR¹²—(C(R¹²)₂)ₙ—H, —NR¹²—(C(R¹²)₂)ₙ—R¹³, —NR¹²—(C(R¹²)₂)ₙ—N(R¹²)₂, —NR¹²—(C(R¹²)₂)—NR¹²—(C(R¹²)₂)ₙ—N(R¹²)₂, NR¹²—(C(R¹²)₂)ₙ—NR¹²—(C(R¹²)₂)ₙ—OR¹²;
n=1-4 at each occurrence, with the proviso that when the carbon chain of length n is between two heteroatoms, n=2-4;
R² and R⁵ are each independently —H, —OH -halo, —CN, —N₃, —NH₂, —NH—C₁-C₆ alkyl, —N(C₁-C₆ alkyl)₂, —C₁-C₆ alkyl, —O—C₁-C₆ alkyl, or —S—C₁-C₆ alkyl, wherein each C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R³ and R⁴ are each independently —H, —OH, -halo, —NH₂, —CN, —NO₂, —COOH, —C₁-C₆ alkyl, —C₂-C₆ alkenyl, —C₂-C₆ alkynyl, —O—C₁-C₆ alkyl, —O—C₂-C₆ alkenyl, —O—C₂-C₆ alkynyl, —C(O)O—C₁-C₆ alkyl, —C(O)NH—C₁-C₆ alkyl, —C(O)N(C₁-C₆ alkyl)₂, —C₁-C₆-alkylene-O—C₁-C₆ alkyl, —O—C₁-C₆-alkylene-O—C₁-C₆ alkyl, —C₁-C₆-alkylene-C(O)O—C₁-C₆ alkyl, —C₁-C₆-alkylene-C(O)NH—C₁-C₆ alkyl, —OC(O)—C₁-C₆ alkyl, —OC(O)—C₂-C₆ alkenyl, —OC(O)—C₂-C₆ alkynyl, —C₁-C₆ alkylene-OC(O)—C₁-C₆ alkyl, —C₁-C₆ alkylene-OC(O)—C₂-C₆ alkenyl, —C₁-C₆ alkylene-OC(O)—C₂-C₆ alkynyl, —S—C₁-C₆ alkyl, —S(O)—C₁-C₆ alkyl, —S(O)₂—C₁-C₆ alkyl, —S(O)₂NH—C₁-C₆ alkyl, —S(O)₂NH—C₂-C₆ alkenyl, —S(O)₂NH—C₂-C₆ alkynyl, —C(O)-benzyl, —C(O)O—C₁-C₆ alkyl, —C(O)—C₁-C₆ alkyl, -phenyl, —O-phenyl, —S-phenyl, -benzyl, —O-benzyl, —CHO, —NHC(O)H, —NHOH, —NH—O—C₁-C₆ alkyl, —NH—C₁-C₆ alkyl, —N(C₁-C₆ alkyl)₂, —NHC(O)—C₁-C₆ alkyl, —N(C₁-C₆ alkyl)(C₂-C₆ alkenyl), —N(C₂-C₆ alkenyl)₂, —NH-phenyl, —NH-benzyl, 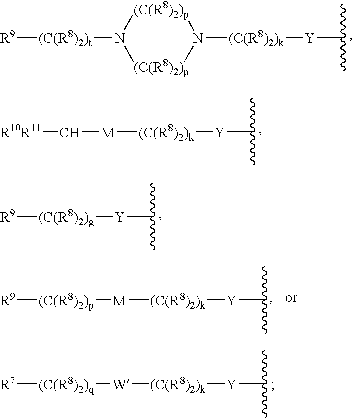
wherein
a=0-1;
g=1-6;
k=0-4;
p=2-4 at each occurrence;
q=0-4;
t=2-4;
M is a bond, —NR⁸—, —O—, —N((C(R⁸)₂)ₚN(R⁸)₂)—, —N((C(R⁸)₂)ₚOR⁸)—, —CC—, —CH═CH—, or phenylene;
W′ is a bond, —N(R⁸)—, —O—, —CC—, —CH═CH—, or phenylene;
Y is —(CH₂)ₐ—, —O—, —S—, —C(O)N(R⁸)—, —C(S)N(R⁸), or —NR⁸—;
when M is phenylene then p=0-4 and r=0-4;
when M is —O— and R⁹ is —OR⁸ then p=1-4;
when M is —CC— or —CH═CH— and Y is —S—, —O— or —NR⁸— then k=1-4;
when M is —CC— or —CH═CH— then p=1-4;
when W′ is —CC— or —CH═CH— and R⁷ is bonded through a heteroatom then q=1-4;
when W′ is a bond and q=0 and R⁷ is bonded through a N atom and Y is —S—, —O— or —NR⁸-then k=2-4;
when W′ is a bond and k=0 and R⁷ is bonded through a N atom and Y is —S—, —O— or —NR⁸-then q=2-4;
when W′ is not a bond, —CC—, —CH═CH—, or phenylene and R⁷ is bonded through a N atom then q=2-4;
when Y is —NR⁸— and R⁹ is —N(R⁸)₂, —N(R⁸)₃⁺ or —NR⁸(OR⁸) then g=2-6;
when Y is —N(R⁸)— and M is —N(R⁸)—, —O—, —N((C(R⁸)₂)ₚN(R⁸)₂— or —N((C(R⁸)₂)ₚ—OR⁸)— then k=2-4;
when Y is —N(R⁸)— and W′ is —N(R⁸)— or —O— then k=2-4;
when Y is —O— and either M or W′ is —O— then k=1-4;
wherein each —C₁-C₆ alkyl, —C₁-C₆ alkylene, —C₂-C₆ alkenyl, —C₂-C₆ alkynyl, -phenyl, -benzyl, or phenylene is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R⁶ is —H, —OH, or —C₁-C₆ alkyl, wherein each —C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R⁷ is -phenyl, a 5- or 6-membered aromatic monocyclic heterocycle which is unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl; or
R⁷ is a 3- to 7-membered non-aromatic monocyclic heterocycle which is unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl, benzoyl, -oxo or -thio,
wherein each R⁷ is independently unsubstituted or mono or di-substituted on a C atom with —R⁸, —N(R⁸)₂, —OR⁸, —(C(R⁸)₂)ᵣOR⁸, or —(C(R⁸)₂)ᵣN(R⁸)₂, or a 3- to 7-membered monocyclic heterocycle,
wherein r=1-6; or
each R⁷ is independently unsubstituted or mono or di-substituted on a saturated C atom with —O(C(R⁸)₂)ᵣO—,
wherein r=1-6; or
each R⁷ is independently unsubstituted or mono-substituted on a N atom with —R⁸;
R⁸ is each independently —H, —C₁-C₆ alkyl, —C₂-C₆ alkenyl, —C₂-C₆ alkynyl, —C₃-C₈ monocyclic cycloalkyl, —C(O)—C₁-C₆ alkyl, —C(O)O—C₁-C₆ alkyl, or -phenyl, wherein each —C₁-C₆ alkyl —C₂-C₆ alkenyl, —C₂-C₆ alkynyl, —C₃-C₈ monocyclic cycloalkyl or -phenyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R⁹ is -halo, —N(R⁸)₂, —OR⁸, —N(R⁸)₃⁺, or —NR⁸(OR⁸);
R¹⁰ and R¹¹ are each independently —(C(R⁸)₂)ₛN(R⁸)₂ or —(C(R⁸)₂)ₛOR⁸,
wherein s=1-4;
R¹² is each independently —H, —C₁-C₆ alkyl, —C₂-C₆ alkenyl, —C₂-C₆ alkynyl, —C₃-C₈ monocyclic cycloalkyl, —C₃-C₈ monocyclic cycloalkenyl, -phenyl, -benzyl, a 5- or 6-membered aromatic monocyclic heterocycle, or a 3- to 7-membered non-aromatic monocyclic heterocycle which is unsubstituted or substituted with one or more of —OH, -halo, —NH₂, —CN, —C₁-C₆ alkyl, -oxo or -thio, wherein each —C₁-C₆ alkyl, —C₂-C₆ alkenyl, —C₂-C₆ alkynyl, —C₃-C₈ monocyclic cycloalkyl, -phenyl, or benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹³ is a 5- or 6-membered aromatic monocyclic heterocycle which is unsubstituted or substituted with one or more of —OH, -halo, —NH₂, —CN or —C₁-C₆ alkyl, a 3- to 7-membered non-aromatic monocyclic heterocycle which is unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl, benzoyl, -oxo or -thio, or a 8- to 12-membered bicyclic heterocycle which is unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl, benzoyl, -oxo or -thio,
wherein each R¹³ is independently unsubstituted or mono- or di-substituted with 4-(2,3-dihydro-indol-1-yl), 4-(1,3-dihydro-isoindol-2-yl), —NH-benzoyl, a 5- or 6-membered aromatic monocyclic heterocycle, a 3- to 7-membered non-aromatic monocyclic heterocycle, 8- to 12-membered bicyclic heterocycle, —O-5- or 6-membered aromatic monocyclic heterocycle, —O-3- to 7-membered non-aromatic monocyclic heterocycle, —O-8- to 12-membered bicyclic heterocycle, or R¹⁴,
wherein each -benzoyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹⁴ is —H, -halo, —OH, —CN, —NH₂, —NO₂, —N₃, —COOH, —C₁-C₆ alkyl, —C₂-C₆ alkenyl, —C₂-C₆ alkynyl, —N₃, —NHC(O)—C₁-C₆ alkyl, —C₁-C₆ alkyl —O—C₁-C₆ alkyl, —C₁-C₆ alkylene-C(O)O—C₁-C₆ alkyl, —O—C₁-C₆ alkyl, —S—C₁-C₆ alkyl, —C(O)O—C₁-C₆ alkyl, —C(O)—C₁-C₆ alkyl, -phenyl, —O-phenyl, —S-phenyl, -benzyl, -benzoyl, —C₁-C₆ alkylene-C(O)O-phenyl, —NH—C₁-C₆ alkyl, —N(C₁-C₆ alkyl)₂, —NH-phenyl, —NH-benzyl, —NH—C(O)—C₁-C₆ alkyl, —NH—C(O)—C₂-C₆ alkenyl, —NH—C(O)—C₂-C₆ alkynyl, —C₁-C₆ alkylene-COOH, —C₁-C₆ alkylene-CHO, —C₁-C₆ alkylene-NH—C₁-C₆ alkyl, —C₁-C₆ alkylene-N(C₁-C₆ alkyl)₂, —C(O)NH—C₁-C₆ alkyl, —C(O)N(C₁-C₆ alkyl)₂, —O—C₁-C₆ alkylene-NH—C₁-C₆ alkyl, —O—C₁-C₆ alkylene-N(C₁-C₆ alkyl)₂, —OS(O)₂—C₁-C₆ alkyl, or —SH, wherein each —C₁-C₆ alkyl, —C₁-C₆ alkylene, —C₂-C₆ alkenyl, —C₂-C₆ alkynyl, -phenyl, -benzyl, or benzoyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl,
which comprises reacting a compound of Formula (II) 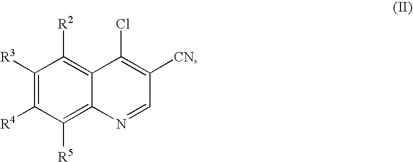
wherein R², R³, R⁴ and R⁵ are as defined above,
with a compound of Formula (III) 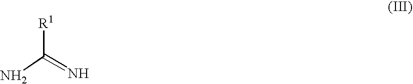
wherein R¹ is as defined above.

24. The process of claim 23, further comprising using a catalyst.

25. The process of claim 24, wherein the catalyst is a base.

26. The process of claim 23, wherein the compound of Formula (III) 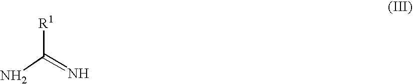
is prepared by a process comprising reacting a compound of Formula (IV)
H—R¹ (IV)
with a compound of Formula (V) 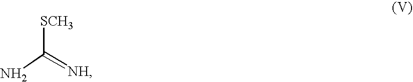
wherein R¹ is as defined in claim 23.
